SEQUENCE LISTING (109 sequences)

SEQ ID NO: 1 - protein, 119 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 2 - protein, 119 aa, synthetic construct
QVQLVETGGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGQGTQVTVSS

SEQ ID NO: 3 - protein, 119 aa, synthetic construct
RVQLVETGGGLVQAGGSLRLSCAVSGSTFSHSAGGWYRQAPEKQRELVAGIRSDGNINYADSVKDRFTISRDNASNTMYLQMNNLKPEDTAVYVCNIRGSYGNTYYSRWGQGAQVTVSS

SEQ ID NO: 4 - protein, 119 aa, synthetic construct
QVQLVETGGGLVQAGGSLRLSCAASGSTFSHSAGGWYRQAPEKQRELVAGIGSDGNINYADSVKDRFTISRDNASNTMYLQMNNLKPEDTAVYVCNIRGSYGNTYYSRWGQGTQVTVSS

SEQ ID NO: 5 - protein, 119 aa, synthetic construct
QVQLVGTGGGLVQAGGSLRLSCAASGSTFSHSAGGWYRQAPEKQRELVAGISSAGNINYADSVKDRFTISRDNASNTMYLQMNNLKPEDTAVYVCNIRGSYGNTYYSRWGQGTQVTVSS

SEQ ID NO: 6 - protein, 119 aa, synthetic construct
QVQLVETGGGLVQAGGSLRLSCAASGSTFSHSAGGWYRQAPEKQRELVAGVRSDGNINYADSVKDRFTISRDNASNTMYLQMNNLKPEDTAVYVCNIRGSYGNTYYSRWGQGIQVTVSS

SEQ ID NO: 7 - protein, 119 aa, synthetic construct
QVQLVETEGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGQGTQVTVSS

SEQ ID NO: 8 - protein, 119 aa, synthetic construct
QVQLVETGGGLVQAGGSLRLSCAVSGSTFSHSAGGWYRQAPEKQRELVAGIRSDGNINYADSVKDRFTISRDNASNTMYLQMNNLKPEDTAVYVCNIRGSYGNTYYSRWGQGTQVTVSS

SEQ ID NO: 9 - protein, 119 aa, synthetic construct
QVQLVETRGGLVQAGGSLRLSCAVSGSTFSHSAGGWYRQAPEKQRELVAGIGSAGNINYADSVKDRFTISRDNASNTMYLQMNNLKPEDTAVYVCNIRGSYGNTYYSRWGQGTQVTVSS

SEQ ID NO: 10 - protein, 119 aa, synthetic construct
QVQLVEAGGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGQGIQVTVSS

SEQ ID NO: 11 - protein, 119 aa, synthetic construct
RVQLVETGGGLVQAGGSLRLSCAVSGRTFSHSAGGWYRQAPEKQRELVAGIRSDGNINYADSVKDRFTISRDNASNTMYLQMNNLKPGDTAVYVCNIRGSYGNTYYSRWGQGTQVTVSS

SEQ ID NO: 12 - protein, 119 aa, synthetic construct
RVRLVETGGGLVQAGGSLRLSCAASGSTFSHSAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 13 - protein, 119 aa, synthetic construct
QVQLVGTGGGLVQAGGSLRLSCAASGSTFSHNAGGWYRQAPEKQRELVAGISSDGNINYADSVKDRFTISRDNASNTMYLQMNNLKPEDAAVYVCNIRGSYGNTYYSRWGQGTQVTVSS

SEQ ID NO: 14 - protein, 119 aa, synthetic construct
QVRLVETGGGLVQAGGSLRLSCAASGSTFSHSAGGWYRQAPEKQRELVAGISSDGNINYADSVKDRFTISRDNASNTMYLQMNNLKPEDTAVYVCNIRGSYGNTYYSRWGQGTQVAVSS

SEQ ID NO: 15 - protein, 119 aa, synthetic construct
QVQLVETRGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 16 - protein, 119 aa, synthetic construct
QVQLVGTGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTRVTVSS

SEQ ID NO: 17 - protein, 119 aa, synthetic construct
RVQLVETRGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 18 - protein, 119 aa, synthetic construct
RVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGIQVTVSS

SEQ ID NO: 19 - protein, 119 aa, synthetic construct
QVQLVGTGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 20 - protein, 119 aa, synthetic construct
QVQLVETGGGLVQAGGSLRLSCAASGSTFSHYAGGWYRQAPEKQRELVAGISSDGNINYADSVKDRFTISRDNASNTMYLQMNNLKPGDTAVYVCNIRGSYGNTYYSRWGRGTQVTVSS

SEQ ID NO: 21 - protein, 119 aa, synthetic construct
QVQLVETGGGLVQAGGSLRLSCAASGSTFSHYAGGWYRQAPEKQRELVAGISSDGNINYADSVKDRFTISRDNASNTMYLQMNNLKPEDTAVYICNIRGSYGNTYYRRWGQGTQVTVSS

SEQ ID NO: 22 - protein, 119 aa, synthetic construct
QVQLVETEGGLVQAGGSLRLSCAASGRTFSHNAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 23 - protein, 119 aa, synthetic construct
QVRLVETGGGLVQAGGSLRLSCAASGSTFSHSAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 24 - protein, 119 aa, synthetic construct
QVQLVETGGGLVQAGGSLRLSCAASGSTFSHKAGGWYRQAPEKQRELVARISSDGNINYADSVKDRFTISRDNASNTMYLQMNNLKPEDTAVYVCNIRGSYGNTYYSRWGQGTQVTVSS

SEQ ID NO: 25 - protein, 119 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQRTQVTVSS

SEQ ID NO: 26 - protein, 119 aa, synthetic construct
QVRLVETGGGLVQAGGSLRLSCAASGSTFSHNAGGWYRQAPEKQRELVAGINSYGNINYADSVKDRFTISRDNASNTMYLQMNNLKPEDTAVYVCNIRGSYGNTYYSRWGQGTQVTVSS

SEQ ID NO: 27 - protein, 119 aa, synthetic construct
QVQLVKTGGGLVQAGGSLRLSCAASGSTFSHNAGGWYRQAPEKQRELVAGISSDGNINYADSVKDRFTISRDNASNTMYLQMNNLKPEDTAVYVCNIRGTYGNTYYSRWGQGTQVTVSS

SEQ ID NO: 28 - protein, 119 aa, synthetic construct
QVQLVGTGGGLVQAGGSLRLSCAVSGSTFSHNAGGWYRQAPEKQRELVAGISSDGNINYADSVKDRFTISRDNASNTMHLQMNNLKPEDTAVYVCNIRGSYGNTYYSRWGQGTQVTVSS

SEQ ID NO: 29 - protein, 119 aa, synthetic construct
QVQLVETRGGLVQAGGSLRLSCAASGITFSHNAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 30 - protein, 119 aa, synthetic construct
QVQLVGTGGGLVQAGGSLRLSCAASGSTFSHNAGSWYRQAPEKQRELVAGIRSDGNINYADSVKDRFTISRDNASNTMYLQMNNLKPEDTAVYVCNIRGSYGNTYYSRWGQGTQVTVSS

SEQ ID NO: 31 - protein, 119 aa, synthetic construct
RVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQRTQVTVSS

SEQ ID NO: 32 - protein, 119 aa, synthetic construct
RVQLVETGGGLVQAGGSLRLSCAASGSTFSHSAGGWYRQAPEKQRELVAGISSDGNINYADSVKDRFTISRDNASNTMYLQMNNLKPEDTAVYVCNIRGSYGNTYYSRWGQGTRVTVSS

SEQ ID NO: 33 - protein, 119 aa, synthetic construct
QVRLVETGGGLVQAGGSLRLSCAVSGSTFSHSAGGWYRQAPEKQRELVAGISSAGNINYADSVKDRFTISRDNASNTMYLQMNNLKPEDTAVYVCNIRGSYGNTYYSRWGQGTQVTVSS

SEQ ID NO: 34 - protein, 119 aa, synthetic construct
QVQLVGTGGGLVQAGGSLRLSCAASGSTFSHSAGGWYRQAPGKQRELVAGIRSDGNINYADSVKDRFTISRDNASNTMYLQMNNLRPEDTAVYVCNIRGSYGNTYYSRWGQGTQVTVSS

SEQ ID NO: 35 - protein, 119 aa, synthetic construct
QVQLVETGGGLVQAGGSLRLSCAASGSTFSHSAGGWYRQAPEKQRELVAGIRSDGNINYADSVKDRFTISRDNASNTMYLQMNNLKPEDTAVYVCNIRGSYGSTYYSRWGQGTQVTVSS

SEQ ID NO: 36 - protein, 119 aa, synthetic construct
QVQLVETGGGLVQAGGSLRLSCAASGSTFSHSAGGWYRQAPEKQRELVAGISSAGNINYADSVKDRFTISRDNASNTMYLQMNNLKPEDTAVYVCNIRGSYGNTYYSRWGQRTQVTVSP

SEQ ID NO: 37 - protein, 119 aa, synthetic construct
QVQLVETGGGLVQAGGSLRLSCAASGSTFSHSAGGWYRQAPEKQRELVAGIRSDGNINYADSVKDRFTISRDNASNTMYLQMDNLKPEDTAVYVCNIRGSYGNTYYSRWSQGTQVTVSS

SEQ ID NO: 38 - protein, 119 aa, synthetic construct
RVQLVETGGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGQGTQVTVSS

SEQ ID NO: 39 - protein, 119 aa, synthetic construct
QVQLVGTGGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGQGTQVTVSS

SEQ ID NO: 40 - protein, 119 aa, synthetic construct
RVQLVETGGGLVQAGGSLRLSCAASGSTFSHSAGGWYRQAPEKQRELVAGISSAGNINYADSVKDRFTISRDNASNTMYLQMNNLKPEDTAVYVCNIRGSYGNTYYSRWGQGIQVTVSS

SEQ ID NO: 41 - protein, 119 aa, synthetic construct
RVQLVETGGGLVQAGGSLRLSCAASGSTFSHSAGGWYRQAPEKQRELVAGISSAGNINYADSVKDRFTISKDDASNTMYLQMNNLKPEDTAVYVCNIRGSYGNTYYSRWGQGTQVTVSS

SEQ ID NO: 42 - protein, 119 aa, synthetic construct
QVQLVETGGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGRGAQVTVSS

SEQ ID NO: 43 - protein, 119 aa, synthetic construct
QVQLVETGGGLVQAGGSLRLSCAVSGSTFSHSAGGWYRQAPEKQRELVAGIRSDGNINYADSVKDRFTISRGNASNTMYLQMNNLKPEDTAVYVCNIRGSYGNTYYSRWGQGTQVTVSS

SEQ ID NO: 44 - protein, 119 aa, synthetic construct; identical to SEQ ID NO: 7

SEQ ID NO: 45 - protein, 119 aa, synthetic construct
RVQLVETEGGLVQAGGSLRLSCAASGSTFSHSAGGWYRQAPEKQRELVAGIGSDGNINYADSVKDRFTISRDNASNTMYLQMNNLKPEDTAVYVCNIRGSYGNTYYSRWGQGTQVTVSS

SEQ ID NO: 46 - protein, 119 aa, synthetic construct
RVQLVETGGGLVQAGGSLRLSCAASGSTFSHSAGGWYRQAPEKQRELVAGIRSDGNINYADSVKGRFTISRDNASNTMYLQMNNLKPEDTAVYVCNIRGSYGNTYYSRWGQGIQVTVSS

SEQ ID NO: 47 - protein, 119 aa, synthetic construct
QVRLVETGGGLVQAGGSLRLSCAASGSTFSHSAGGWYRQAPEKQRELVVGIRSDGNISYADSVKDRFTISRDNASNTMYLQMNNLKPEDTAVYVCNIRGSYGNTYYSRWGQGTQVTVSS

SEQ ID NO: 48 - protein, 119 aa, synthetic construct
QVQLVETGGGLVQAGGSLRLSCAASGSTFSHSAGGWYRQAPEKQRELVAGIRSDGNINYADSVKGRFTISRDNASNTMYLQMNNLKPEDTAVYVCNIRGSYGNTYYSRWGQRTQVTVSS

SEQ ID NO: 49 - protein, 119 aa, synthetic construct
RVQLVETGGGLVQAGGSLRLSCAASGNTFSHSAGGWYRQAPEKQRELVAGIRSDGNISYADSVKDRFTISRDNASNTMYLQMNNLKPEDTAVYVCNIRGSYGNTYYSRWGQGTQVTVSS

SEQ ID NO: 50 - protein, 119 aa, synthetic construct
QVQLVETGGGLVQAGGSLRLSCAVSGSTFSHSAGGWYRQAPEKQRELVAGIRSDGNINYADSVKDRLTISRDNASNTMYLQMNNLKPEDTAVYVCNIRGSYGNTYYSRWGQGTQVTVSS

SEQ ID NO: 51 - protein, 119 aa, synthetic construct
RVQLVETGGGLVQAGGSLRLSCAVSGRTFSHSAGGWYRQAPEKQRELVAGIRSDGNINYADSVKDRFTISRDNASNTMYLQMNNLKPGDTGVYVCNIRGSYGNTYYSRGGQGTQVTVFS

SEQ ID NO: 52 - protein, 119 aa, synthetic construct
RVQLVETGGGLVQAGGSLRLSCAASGSTFSHSAGGWYRQAPEKQRELVAGIGSDGNINYADSVKDRFTISRDNASNTMYLQMNNLKPEDTAVYVCNIRGSYGNTYYSRWGQGTQVTVSS

SEQ ID NO: 53 - protein, 119 aa, synthetic construct
RVQLVETGGGLVQAGGSLRLSCAVSGRTFSHSAGGWYRQAPEKQRELVAGIRSDGNINYADSVKDRFTISRDNASNTMYLQMNNLKPGDTAVYVCNIRGSYGNTYYSRWGQGTQVTVFS

SEQ ID NO: 54 - protein, 10 aa, synthetic construct
GSTFSHNAGG

SEQ ID NO: 55 - protein, 9 aa, synthetic construct
GISSDGNIN

SEQ ID NO: 56 - protein, 13 aa, synthetic construct
NIRGSYGNTYYSR

SEQ ID NO: 57 - protein, 10 aa, synthetic construct
GSTFSHSAGG

SEQ ID NO: 58 - protein, 9 aa, synthetic construct
GIRSDGNIN

SEQ ID NO: 59 - protein, 9 aa, synthetic construct
GIGSDGNIN

SEQ ID NO: 60 - protein, 9 aa, synthetic construct
GISSAGNIN

SEQ ID NO: 61 - protein, 9 aa, synthetic construct
GVRSDGNIN

SEQ ID NO: 62 - protein, 9 aa, synthetic construct
GIGSAGNIN

SEQ ID NO: 63 - protein, 10 aa, synthetic construct
GRTFSHSAGG

SEQ ID NO: 64 - protein, 25 aa, synthetic construct
QVQLVETGGGLVQAGGSLRLSCAAS

SEQ ID NO: 65 - protein, 11 aa, synthetic construct
WGQGTQVTVSS

SEQ ID NO: 66 - protein, 25 aa, synthetic construct
RVQLVETGGGLVQAGGSLRLSCAVS

SEQ ID NO: 67 - protein, 11 aa, synthetic construct
WGQGAQVTVSS

SEQ ID NO: 68 - protein, 25 aa, synthetic construct
QVQLVGTGGGLVQAGGSLRLSCAAS

SEQ ID NO: 69 - protein, 11 aa, synthetic construct
WGQGIQVTVSS

SEQ ID NO: 70 - protein, 25 aa, synthetic construct
QVQLVETEGGLVQAGGSLRLSCAAS

SEQ ID NO: 71 - protein, 25 aa, synthetic construct
QVQLVETGGGLVQAGGSLRLSCAVS

SEQ ID NO: 72 - protein, 25 aa, synthetic construct
QVQLVETRGGLVQAGGSLRLSCAVS

SEQ ID NO: 73 - protein, 25 aa, synthetic construct
QVQLVEAGGGLVQAGGSLRLSCAAS

SEQ ID NO: 74 - protein, 289 aa, synthetic construct
QVQLVETGGDLVQPGGSLRLSCAASGLTLDYYAIGWVRQAPGKEREGVSCITPQDGNTYYDDSVMGRFTILRDNAKNMVYLQMNNLKPEDTAVYFCAAAGALTLDPSEYEYWGQGTQVTVSSGGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQLPETGGHHHHHH

SEQ ID NO: 75 - protein, 286 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQLPETGGHHHHHH

SEQ ID NO: 76 - protein, 286 aa, synthetic construct
QVQLVETGGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGQGTQVTVSSGGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQLPETGGHHHHHH

SEQ ID NO: 77 - protein, 286 aa, synthetic construct
RVQLVETGGGLVQAGGSLRLSCAVSGSTFSHSAGGWYRQAPEKQRELVAGIRSDGNINYADSVKDRFTISRDNASNTMYLQMNNLKPEDTAVYVCNIRGSYGNTYYSRWGQGAQVTVSSGGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQLPETGGHHHHHH

SEQ ID NO: 78 - protein, 669 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGSGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSQVQLVETGGGLVQAGGSLRLSCAASGSTFSHSAGGWYRQAPEKQRELVAGIRSDGNINYADSVKDRFTISRDNASNTMYLQMNNLKPEDTAVYVCNIRGSYGNTYYSRWGQGTQVTVSSGGLPETGGHHHHHH

SEQ ID NO: 79 - protein, 669 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGSGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSRVQLVETGGGLVQAGGSLRLSCAVSGSTFSHSAGGWYRQAPEKQRELVAGIRSDGNINYADSVKDRFTISRDNASNTMYLQMNNLKPEDTAVYVCNIRGSYGNTYYSRWGQGAQVTVSSGGLPETGGHHHHHH

SEQ ID NO: 80 - protein, 669 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGSGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSQVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGLPETGGHHHHHH

SEQ ID NO: 81 - protein, 672 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGSGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSQVQLVETGGDLVQPGGSLRLSCAASGLTLDYYAIGWVRQAPGKEREGVSCITPQDGNTYYDDSVMGRFTILRDNAKNMVYLQMNNLKPEDTAVYFCAAAGALTLDPSEYEYWGQGTQVTVSSGGLPETGGHHHHHH

SEQ ID NO: 82 - protein, 301 aa, synthetic construct
QVQLVETGGDLVQPGGSLRLSCAASGLTLDYYAIGWVRQAPGKEREGVSCITPQDGNTYYDDSVMGRFTILRDNAKNMVYLQMNNLKPEDTAVYFCAAAGALTLDPSEYEYWGQGTQVTVSSGGGGGSCDLPQTHNLRNKRALTLLVQMRRLSPLSCLKDRKDFRFPQEKVDAQQIQNAQAIPVLQELTQQVLNIFTSKDSSAAWDASLLDSFCNDLHQQLNDLKACVMQEVGVQEPPLTQEDYLLAVRTYFHRITVYLREKKHSPCAWEVVRAEVWRAMSSSAKLLARLSEEKEHHHHHH

SEQ ID NO: 83 - protein, 298 aa, synthetic construct
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSSGGGGGSCDLPQTHNLRNKRALTLLVQMRRLSPLSCLKDRKDFRFPQEKVDAQQIQNAQAIPVLQELTQQVLNIFTSKDSSAAWDASLLDSFCNDLHQQLNDLKACVMQEVGVQEPPLTQEDYLLAVRTYFHRITVYLREKKHSPCAWEVVRAEVWRAMSSSAKLLARLSEEKEHHHHHH

SEQ ID NO: 84 - protein, 298 aa, synthetic construct
RVQLVETGGGLVQAGGSLRLSCAVSGSTFSHSAGGWYRQAPEKQRELVAGIRSDGNINYADSVKDRFTISRDNASNTMYLQMNNLKPEDTAVYVCNIRGSYGNTYYSRWGQGAQVTVSSGGGGGSCDLPQTHNLRNKRALTLLVQMRRLSPLSCLKDRKDFRFPQEKVDAQQIQNAQAIPVLQELTQQVLNIFTSKDSSAAWDASLLDSFCNDLHQQLNDLKACVMQEVGVQEPPLTQEDYLLAVRTYFHRITVYLREKKHSPCAWEVVRAEVWRAMSSSAKLLARLSEEKEHHHHHH

SEQ ID NO: 85 - protein, 25 aa, synthetic construct
RVQLVETGGGLVQAGGSLRLSCAAS

SEQ ID NO: 86 - protein, 25 aa, synthetic construct
QVRLVETGGGLVQAGGSLRLSCAVS

SEQ ID NO: 87 - protein, 25 aa, synthetic construct
RVQLVETEGGLVQAGGSLRLSCAAS

SEQ ID NO: 88 - protein, 25 aa, synthetic construct
QVRLVETGGGLVQAGGSLRLSCAAS

SEQ ID NO: 89 - protein, 25 aa, synthetic construct
QVQLVETRGGLVQAGGSLRLSCAAS

SEQ ID NO: 90 - protein, 25 aa, synthetic construct
RVQLVETRGGLVQAGGSLRLSCAAS

SEQ ID NO: 91 - protein, 25 aa, synthetic construct
QVQLVGTGGGLVQAGGSLRLSCAVS

SEQ ID NO: 92 - protein, 11 aa, synthetic construct
WSQGTQVTVSS

SEQ ID NO: 93 - protein, 11 aa, synthetic construct
WGRGAQVTVSS

SEQ ID NO: 94 - protein, 11 aa, synthetic construct
WGQGTQVTVFS

SEQ ID NO: 95 - protein, 11 aa, synthetic construct
WGQGTRVTVSS

SEQ ID NO: 96 - protein, 11 aa, synthetic construct
GGQGTQVTVFS

SEQ ID NO: 97 - protein, 11 aa, synthetic construct
WGQRTQVTVSS

SEQ ID NO: 98 - protein, 13 aa, synthetic construct
NIRGSYGSTYYSR

SEQ ID NO: 99 - protein, 13 aa, synthetic construct
NIRGSYGNTYYRR

SEQ ID NO: 100 - protein, 13 aa, synthetic construct
NIRGTYGNTYYSR

SEQ ID NO: 101 - protein, 9 aa, synthetic construct
GIRSDGNIS

SEQ ID NO: 102 - protein, 10 aa, synthetic construct
GNTFSHSAGG

SEQ ID NO: 103 - protein, 10 aa, synthetic construct
GITFSHNAGG

SEQ ID NO: 104 - protein, 10 aa, synthetic construct
GSTFSHNAGS

SEQ ID NO: 105 - protein, 10 aa, synthetic construct
GRTFSHNAGG

SEQ ID NO: 106 - protein, 10 aa, synthetic construct
GSTFSHKAGG

SEQ ID NO: 107 - protein, 10 aa, synthetic construct
GSTFSHYAGG

SEQ ID NO: 108 - protein, 9 aa, synthetic construct
RISSDGNIN

SEQ ID NO: 109 - protein, 9 aa, synthetic construct
GINSYGNIN

(19) United States
(12) Patent Application Publication
(10) Pub. No.: US 2023/0295284 A1
(43) Pub. Date: Sep. 21, 2023

(54) ENHANCED-AFFINITY ANTI-ECM NANOBODY-CYTOKINE FUSIONS AND THEIR APPLICATIONS

(71) Applicant: Massachusetts Institute of Technology, Cambridge, MA (US)

(72) Inventors: Richard O. Hynes, Winchester, MA (US); Karl Dane Wittrup, Chestnut Hill, MA (US); Noor Jailkhani, Cambridge, MA (US); Noor Momin, Cambridge, MA (US); Emi Lutz, Cambridge, MA (US)

(73) Assignee: Massachusetts Institute of Technology, Cambridge, MA (US)

(21) Appl. No.: 18/162,512

(22) Filed: Jan. 31, 2023

Related U.S. Application Data
(60) Provisional application No. 63/304,954, filed on Jan. 31, 2022.

(51) Int. Cl. C07K 16/18; A61K 47/68; C07K 14/54; C07K 14/55; C07K 14/56; C07K 14/565; C07K 14/57; C07K 14/525; A61P 35/00

(52) U.S. Cl. CPC C07K 16/18; A61K 47/6843; A61K 47/6813; C07K 14/5412; C07K 14/5421; C07K 14/5428; C07K 14/5434; C07K 14/55; C07K 14/56; C07K 14/565; C07K 14/57; C07K 14/525; A61P 35/00; C07K 2317/569; C07K 2319/00

(57) ABSTRACT

Provided herein are anti-FN-EIIIB nanobodies, conjugates thereof, and methods of using the nanobodies and conjugates thereof.

### RELATED APPLICATIONS

This application claims priority under 35 U.S.C. § 119(e) to U.S. Provisional Patent Application No. 63/304,954, filed Jan. 31, 2022, which is hereby incorporated by reference in its entirety.

### FEDERALLY SPONSORED RESEARCH

This invention was made with government support under W81XWH-14-1-0240 awarded by the U.S. Army Medical Research and Material Command. The government has certain rights in the invention.

### REFERENCE TO AN ELECTRONIC SEQUENCE LISTING

The contents of the electronic sequence listing (M065670517US01-SEQ-GIC.xml; Size: 106,337 bytes; and Date of Creation: Jan. 30, 2023) is herein incorporated by reference in its entirety.

### BACKGROUND

The extracellular matrix (ECM) and ECM proteins play important physiological roles in growth, wound healing, cell migration, and differentiation, among other processes. Furthermore, in diseased states, the composition of the ECM changes and prior observations indicate that these changes in extracellular matrix (ECM) proteins can play significant roles in cancer progression and metastasis, in cardiovascular diseases and in fibrosis. There is a need for new molecules targeting ECM proteins for use in imaging and targeting disease states.

### SUMMARY

Provided in aspects of the present disclosure are anti-ECM nanobodies coupled to cytokines, which may be used to target the cytokines to tumor microenvironment to stimulate local immune response against the tumor. It is recognized herein that several parameters affect efficacy, including increasing nanobody affinity for tumor ECM, expression level of ECM target, and route of delivery (systemic or intratumoral). The increased affinity of the nanobodies described herein provides a substantial advantage over prior art antibodies, including the “parental NJB2” antibodies. The parental nanobody NJB2 is specific to the EIIIB domain of fibronectin, which shows selective expression in sites of disease such as tumors, metastases and fibroses among others. The increased affinity nanobodies described herein retain their specificity to EIIIB.

In one aspect, the present disclosure provides a nanobody that binds specifically to the EIIIB domain of fibronectin and comprises: a) a complementarity-determining region (CDR)1 comprising G(S/R/I/N)TFSH(N/S/Y/K)AG(G/S); b) a CDR2 comprising (G/R)(I/V)(S/R/G/N)S(D/A/Y)GNI(N/S); and c) a CDR3 comprising NIRG(S/T)YG(N/S)TYY(S/R)R, wherein the nanobody does not comprise SEQ ID NO: 1.

In some embodiments, the nanobody comprises: a) a CDR1 selected from GSTFSHNAGG (SEQ ID NO: 54), GSTFSHSAGG (SEQ ID NO: 57), GSTFSHYAGG (SEQ ID NO: 107), GSTFSHKAGG (SEQ ID NO: 106), GRTFSHNAGG (SEQ ID NO: 105), GSTFSHNAGS (SEQ ID NO: 104), GITFSHNAGG (SEQ ID NO: 103), GRTFSHSAGG (SEQ ID NO: 63), and GNTFSHSAGG (SEQ ID NO: 102); b) a CDR2 selected from GISSDGNIN (SEQ ID NO: 55), GIRSDGNIN (SEQ ID NO: 58), GIGSDGNIN (SEQ ID NO: 59), GISSAGNIN (SEQ ID NO: 60), RISSDGNIN (SEQ ID NO: 108), GINSYGNIN (SEQ ID NO: 109), GIGSAGNIN (SEQ ID NO: 62), GIRSDGNIS (SEQ ID NO: 101), and GVRSDGNIN (SEQ ID NO: 61); and c) a CDR3 selected from NIRGSYGNTYYSR (SEQ ID NO: 56), NIRGTYGNTYYSR (SEQ ID NO: 100), NIRGSYGNTYYRR (SEQ ID NO: 99), and NIRGSYGSTYYSR (SEQ ID NO: 98).

In some embodiments, the nanobody comprises a CDR1, CDR2, and CDR3 comprising: a) GSTFSHSAGG (SEQ ID NO: 57), GIRSDGNIN (SEQ ID NO: 58), and NIRGSYGNTYYSR (SEQ ID NO: 56) respectively; b) GSTFSHSAGG (SEQ ID NO: 57), GIGSDGNIN (SEQ ID NO: 59), and NIRGSYGNTYYSR (SEQ ID NO: 56) respectively; c) GSTFSHSAGG (SEQ ID NO: 57), GISSAGNIN (SEQ ID NO: 60), and NIRGSYGNTYYSR (SEQ ID NO: 56) respectively; d) GSTFSHSAGG (SEQ ID NO: 57), GIGSAGNIN (SEQ ID NO: 62), and NIRGSYGNTYYSR (SEQ ID NO: 56) respectively; e) GSTFSHSAGG (SEQ ID NO: 57), GVRSDGNIN (SEQ ID NO: 61), and NIRGSYGNTYYSR (SEQ ID NO: 56) respectively; or f) GRTFSHSAGG (SEQ ID NO: 63), GIRSDGNIN (SEQ ID NO: 58), and NIRGSYGNTYYSR (SEQ ID NO: 56) respectively.

In some embodiments, the nanobody comprises a CDR1, CDR2, and CDR3 comprising, GSTFSHSAGG (SEQ ID NO: 57), GIRSDGNIN (SEQ ID NO: 58), and NIRGSYGNTYYSR (SEQ ID NO: 56) respectively.

In some embodiments, the nanobody further comprises a framework 1 comprising:

a)
(SEQ ID NO: 64)
QVQLVETGGGLVQAGGSLRLSCAAS;
b)
(SEQ ID NO: 68)
QVQLVGTGGGLVQAGGSLRLSCAAS;
c)
(SEQ ID NO: 85)
RVQLVETGGGLVQAGGSLRLSCAAS;
d)
(SEQ ID NO: 86)
QVRLVETGGGLVQAGGSLRLSCAVS;
e)
(SEQ ID NO: 66)
RVQLVETGGGLVQAGGSLRLSCAVS;
f)
(SEQ ID NO: 71)
QVQLVETGGGLVQAGGSLRLSCAVS;
g)
(SEQ ID NO: 72)
QVQLVETRGGLVQAGGSLRLSCAVS;
h)
(SEQ ID NO: 70)
QVQLVETEGGLVQAGGSLRLSCAAS;
i)
(SEQ ID NO: 87)
RVQLVETEGGLVQAGGSLRLSCAAS;
j)
(SEQ ID NO: 88)
QVRLVETGGGLVQAGGSLRLSCAAS;
k)
(SEQ ID NO: 73)
QVQLVEAGGGLVQAGGSLRLSCAAS;
l)
(SEQ ID NO: 89)
QVQLVETRGGLVQAGGSLRLSCAAS;
m)
(SEQ ID NO: 90)
RVQLVETRGGLVQAGGSLRLSCAAS;
or
n)
(SEQ ID NO: 91)
QVQLVGTGGGLVQAGGSLRLSCAVS.

In some embodiments, the nanobody comprises a framework 1 comprising:

a)
(SEQ ID NO: 66)
RVQLVETGGGLVQAGGSLRLSCAVS;
b)
(SEQ ID NO: 72)
QVQLVETRGGLVQAGGSLRLSCAVS;
c)
(SEQ ID NO: 71)
QVQLVETGGGLVQAGGSLRLSCAVS;
d)
(SEQ ID NO: 70)
QVQLVETEGGLVQAGGSLRLSCAAS;
e)
(SEQ ID NO: 64)
QVQLVETGGGLVQAGGSLRLSCAAS;
or
f)
(SEQ ID NO: 73)
QVQLVEAGGGLVQAGGSLRLSCAAS.

In some embodiments, the nanobody further comprises a framework 4 comprising:

a)
(SEQ ID NO: 65)
WGQGTQVTVSS;
b)
(SEQ ID NO: 69)
WGQGIQVTVSS;
c)
(SEQ ID NO: 92)
WSQGTQVTVSS;
d)
(SEQ ID NO: 97)
WGQRTQVTVSS;
e)
(SEQ ID NO: 93)
WGRGAQVTVSS;
f)
(SEQ ID NO: 67)
WGQGAQVTVSS;
g)
(SEQ ID NO: 94)
WGQGTQVTVFS;
h)
(SEQ ID NO: 95)
WGQGTRVTVSS;
or
i)
(SEQ ID NO: 96)
GGQGTQVTVFS.

In some embodiments, the nanobody comprises a framework 4 comprising:

a)
(SEQ ID NO: 67)
WGQGAQVTVSS;
b)
(SEQ ID NO: 65)
WGQGTQVTVSS;
or
c)
(SEQ ID NO: 69)
WGQGIQVTVSS.

In some embodiments, the nanobody comprises the amino acid sequence of any one of SEQ ID Nos. 2-53. In some embodiments, the nanobody comprises the amino acid sequence of SEQ ID NO: 3.

In some embodiments, the nanobody is conjugated to a cargo molecule. In some embodiments, the cargo molecule is linked to the N-terminus or C-terminus of the nanobody. In some embodiments, the cargo molecule is a molecular imaging probe, a drug, a toxin, an an antibody, a cytokine, an enzyme (e.g., ECM modifying enzyme), a CAR cell such as a CAR-T cell, a radioisotope, a protein, or a nanoparticle. In some embodiments, the nanobody is attached to or associated with a carrier. In some embodiments, the carrier is a nanoparticle. In some embodiments, the carrier further comprises cargo molecules. In some embodiments, the cargo molecule is selected from drugs, toxins, antibodies, cytokines, nucleic acids, siRNAs, shRNAs, modified mRNAs, DNA, oligonucleotides, CRISPR Cas9 reagents, and imaging probes. In some embodiments, the cargo molecule is a cytokine selected from IL-2, IL-6, IL-8, IL-10, IL-12, IL-15, IL-18, TNF, IFNg, IFNb, IFNa, and a chemokine or mutants thereof.

In one aspect, the present disclosure provides a pharmaceutical composition comprising a nanobody of the disclosure and a pharmaceutically acceptable carrier.

In one aspect, the present disclosure provides a nucleic acid comprising a nucleic acid sequence encoding a nanobody of the disclosure. The disclosure also provides vectors comprising the nucleic acids of the disclosure and cells comprising the nucleic acids or vectors of the disclosure. The disclosure further provides a method for producing a nanobody comprising (i) culturing a cell comprising a nucleic acid or vector of the disclosure under conditions suitable for allowing the expression of the nanobody, and (ii) recovering the expressed nanobody.

In one aspect, the disclosure provides a method of treating a disease or condition associated with fibronectin, such as EIIIB-containing fibronectin, comprising administering to a subject in need thereof an effective amount of the nanobody or pharmaceutical composition of the disclosure.

In one aspect, the disclosure provides a method comprising administering an effective amount of the nanobody of the disclosure conjugated to a cargo molecule to a subject having a disease or condition associated with fibronectin, to deliver the cargo molecule to the extracellular matrix of the diseased tissue.

In some embodiments, the method comprises determining the presence or absence of the EIIIB domain of fibronectin in a subject and determining whether the subject has the disease or condition associated with fibronectin.

In some embodiments, the method comprises tracking the progression of the disease or condition associated with fibronectin by measuring the presence or absence of the EIIIB domain of fibronectin over time.

In some embodiments, the method comprises measuring the presence or absence of the EIIIB domain of fibronectin in a tissue sample isolated from a subject at a first time point and a second time point and determining the progression of the disease or condition to an advanced stage based on changes in the presence or absence of the EIIIB domain of fibronectin at the first and second time points, wherein i) when the EIIIB domain of fibronectin is present at a higher level in the isolated tissue sample from the second time point, the disease or condition has progressed to an advanced stage, and ii) when the EIIIB domain of fibronectin is present at a lower level in the isolated tissue sample from the second time point, the disease or condition has regressed to a less advanced stage.

In some embodiments, the disease or condition is a cancer, a cardiovascular disease, an inflammatory disorder, or fibrosis. In some embodiments, the cancer is a metastatic cancer. In some embodiments, the cancer is melanoma, breast cancer or pancreatic cancer.

In some embodiments, the administration is intravenous, intratumoral, subcutaneous, intramuscular, intradermal, intraperitoneal, intranasal, intratracheal, intrathecal, intracranial, intraperitoneal, rectal, nasal, or oral.

In some aspects, a nanobody cytokine fusion protein, comprising a nanobody that binds specifically to the EIIIB domain of fibronectin fused to a cytokine is provided. In some embodiments, the cytokine is selected from the group consisting of IL-2, IL-6, IL-8, IL-10, IL-12, IL-18, TNF, IFN-γ, IFN-β, chemokines, and IFN-α and their mutants. In some embodiments, the nanobody cytokine fusion protein comprises at least two nanobodies fused to the cytokine. In some embodiments, the nanobody cytokine fusion protein comprises 2-25 nanobodies fused to the cytokine. In some embodiments, the nanobodies comprise multiple copies of the same nanobody. In some embodiments, the nanobodies comprise at least 2 different nanobodies.

In some embodiments, the nanobody cytokine fusion protein comprises at least two cytokines fused to the nanobody. In some embodiments, the at least two cytokines are attached to terminal ends of the nanobody. In some embodiments, the at least two cytokines are the same cytokine. In some embodiments, the at least two cytokines are two different cytokines.

In some embodiments, the nanobody cytokine fusion protein further comprises albumin.

### BRIEF DESCRIPTION OF THE DRAWINGS

FIGS. 1A-1D show the effects of an NJB2-IL-2 immunocytokine with nanomolar affinity to EIIIB in B16F10 tumors. (FIG. 1A) Schematic of protein fusions with the nanobody as a rectangle and the IL-2 as an oval. IL2-mt: mutant IL2 inactivated by mutations described by Lombardi et al. Nat. Commun. 8, 15373 (2017) and Liu et al. J. Immunother. 32, 887-894 (2009). (FIG. 1B) NJB2-IL2-mt, NJT6-IL2, and NJB2-IL2 on a non-reducing SDS-PAGE gel with coomassie blue stain. (FIG. 1C) B16F10 study timeline. 1M: 1 million. (FIG. 1D) Tumor growth (left) and survival (right). Tumor area (mean+SD) is shown until a mouse in that group is euthanized. Statistical significance for survival was generated by a log-rank Mantel-Cox test. ns: not significant. N=5 for all groups.

FIGS. 2A-2C show the evolution of nanobodies with picomolar affinity to EIIIB using yeast surface display. (FIG. 2A) Schematic of yeast surface display. (FIG. 2B) The structure of NJB2 was predicted using ABodyBuilder on the SAbPred server. CDRs (gray) and framework (tan) are shown in ribbon format. The location of mutations in LMJ1.2C and LMJ2.5I are colored and shown in sphere format. Graphic generated with UCSF Chimera. (FIG. 2C) The affinities of 10 novel nanobodies generated by yeast surface display determined using bio-layer interferometry. The KD values are indicated.

FIG. 3 shows that nanobodies based on NJB2 are specific to FN-EIIIB. Western blots show that nanobodies sortase-tagged with biotin detect fragments that contain EIIIB i.e., EIIIB-His and FN (7-15)-EIIIB. They do not bind human or murine plasma fibronectin that lacks EIIIB or normal lung ECM.

FIGS. 4A-4D show the effects of IL-2 immunocytokines with nanomolar and picomolar affinity for FN-EIIIB on tumors after intravenous administration. (FIG. 4A) Schematic of protein fusions (top) and B16F10 study timeline (bottom). 1M: 1 million. (FIG. 4B) Survival is shown until a mouse in that group is euthanized. Survival comparisons were generated by a log-rank Mantel-Cox test. ns: not significant. N=7-9 over two independent experiments. (FIG. 4C) 4T1 study timeline. 1M, 1 million. (FIG. 4D) Tumor growth (left) and survival (right). Tumor area (mean+SD) is shown until a mouse in that group is euthanized. Survival comparisons were generated by a log-rank Mantel-Cox test. ns: not significant. N=6-7.

FIGS. 5A-5C show the effects of IL-2 immunocytokines with nanomolar and picomolar affinity for FN-EIIIB on tumors after intratumoral administration. (FIG. 5A) B16F10 study timeline. (FIG. 5B) Tumor growth (left) and survival (right). Tumor area (mean+SD) is shown until a mouse in that group is euthanized. Survival comparisons were generated by a log-rank Mantel-Cox test. ns: not significant. N=8 for TA99+PBS, N=11 for all other groups, over two independent experiments. (FIG. 5C) Results of rechallenge experiment performed on N=1 for NJT6-IL2, N=6 for NJB2-IL2, and N=4 for LMJ2.5I-IL2.

FIGS. 6A-6B show the effects of IL-12 immunocytokines specific for FN-EIIIB after intravenous administration. (FIG. 6A) 4T1 study timeline and dosing details of IL-12 based immunocytokines in the 4T1 model. (FIG. 6B) Survival (left) and tumor growth (right). Tumor area (mean+SD) is shown until a mouse in that group is euthanized. Survival comparisons were generated by a log-rank Mantel-Cox test. ns: not significant. N=6. IP: intra-peritoneal. IV: intravenous.

FIGS. 7A-7B shows NJT6-IFNα, NJB2-IFNα, and LMJ2.5I-IFNα on a non-reducing SDS-PAGE gel with coomassie blue stain. FIG. 7B shows that all IFNα fusion proteins are active in vitro as tested by a RAW Lucia macrophage activation assay.

### DETAILED DESCRIPTION

The present disclosure provides nanobodies that bind the EIIIB domain of fibronectin. These nanobodies are referred to herein as “anti-FN-EIIIB nanobodies.” The present disclosure also provides nanobody conjugates, nucleic acids encoding anti-FN-EIIIB nanobodies or conjugates thereof, compositions comprising the anti-FN-EIIIB nanobodies or conjugates thereof, methods of producing the nanobodies (e.g., recombinant production methods), and methods of using the nanobodies or conjugates thereof, such as methods of treating a disease or condition associated with fibronectin.

Nanobodies that are highly specific to an epitope in the EIIIB domain of fibronectin (FN), which is present in the ECM of diseased tissues but not in normal, healthy adult tissues, were developed. Therefore, the nanobodies generated may be used for in vivo and in vitro diagnostic imaging (such as, but not limited to, immunofluorescence (IF), immunohistochemistry (IHC), positron emission tomography/computed tomograph (PET/CT)) and for targeted therapeutics across a broad spectrum of diseases. Therefore, the nanobodies of the disclosure are useful for both identifying the diseased tissue (and diseased state of the subject) as well as to deliver therapeutics or other cargo to the region.

As shown in the examples, a marked increase in affinity of the new evolved nanobodies based on the parental anti-FN-EIIIB nanobody, NJB2, was observed. Studies with nanobody-cytokine fusions are also described herein. FN-EIIIB-specific NJB2-based immunocytokines (IL2 and IL12) mildly increased survival compared to non-targeting and size control NJT6 via systemic dosing in models of melanoma and breast cancer. Intra-tumoral administration of NJB2-IL2 significantly increased cures compared to non-targeting NJT6 in a model of melanoma. The IL2 fusions of the evolved nanobodies tested in vivo showed trends that suggest that enhancement of affinity can provide benefit. With cytokines, there is the potential that the nanobody-cytokine fusion also binds to other non-disease sites where the cytokine-receptor is expressed, sometimes referred to as the “systemic cytokine sink”. Without wishing to be bound by theory, this will not be the case when other drugs or toxins are attached to these higher affinity binders. Thus, in applications such as nanobody-drug conjugates or nanobody-isotope conjugates, the higher affinity versions are expected to be more beneficial than the parent NJB2.

The higher affinity anti-FN-EIIIB nanobodies of the disclosure have several advantages.
1. Advantage of higher affinity anti-FN-EIIIB nanobodies. One potential way to enhance tumor-specific binding of a nanobody-drug conjugate is to increase the binding affinity of the nanobody to its target. These higher affinity binders can more effectively home to the disease ECM and stay bound for longer durations, potentially increasing the therapeutic effect. These may also help circumvent the problem of limited dosing since smaller amounts of nanobody-drug conjugate may be able to bring about effective local drug concentrations and further reduce systemic toxicity. In the context of nanobody-cytokine fusions, this can be a significant advantage. The biodistribution of an immunocytokine is dependent on (i) the affinity of the nanobody for its target antigen, (ii) the affinity of the cytokine for its receptor and also on (iii) the sites of expression of the target antigen and the cytokine receptors in different cells/organs. This may result in the nanobody-cytokine fusion binding to the target antigen (at the tumor) and also to other non-disease sites where the cytokine-receptor is expressed, sometimes referred to as the “systemic cytokine sink” (3). Higher affinity nanobodies may help outcompete the binding of the cytokine for its receptor, reducing binding in the systemic sink and allowing higher localization at the disease site. In some embodiments, the balance between the target and sink is altered by decreasing the binding of the immunocytokine to the sink by reducing the affinity of the cytokine for its receptor.
The anti-FN-EIIIB nanobodies of the disclosure are specific to proteins/domains that are expressed specifically in diseased tissues such as cancer and are largely absent from normal adult human tissues. Cargo molecules can be site specifically attached to these nanobodies for targeted delivery to diseased sites. This targeted approach offers advantages over systemic therapies, by allowing selective accumulation in tumor and disease ECM. This results in increasing specificity and sensitivity of detection/treatment and reducing background and off-target toxicity.
2. Advantage over other targeted approaches. The nanobodies of the disclosure also have several advantages over other targeted approaches. While other targeted therapies exist, they are often challenged by systemic toxicities due to wider expression of the targeted proteins (4). The selective expression of the EIIIB domain in diseased sites and specific binding of the nanobodies to their target proteins allows for an improvement over other targeting approaches. The rapid clearance of the small (15 kDa) nanobodies also provides an advantage in reduced systemic effects.
3. Wide application in multiple cancer types and disease states (not cancer type restricted. The applications of existing targeted therapies such as HER2-specific nanobodies for breast and ovarian cancer, rituximab for lymphoid tumors, etc., are restricted to cancer type. Therapies and detection tools developed using the anti-FN-EIIIB nanobodies of the disclosure will have the advantage of being usable as pan-cancer therapeutic/targeting agents, since they recognize antigens whose expression is not restricted to a cancer type. In addition to cancer, the target antigen is expressed in other disease states characterized by ECM remodeling. These include, but are not limited to, cardiovascular disorders such as atheroma, aneurysms, myocardial infarction, fibroses, autoimmune and inflammatory disorders. In some embodiments, LC-MS/MS data has been shown to define a signature of ECM proteins abundant in diverse human tumors and metastases as well as ECM proteins with more restricted tumor-type expression. These proteomicly-defined ECM proteins can be explored as additional novel targets.
4. Advantages of targeting the tumor ECM over targeting tumor cells. Targeting extracellular matrix proteins such as the EIIIB domain of FN offers several advantages over existing methods. Tumor-cell targeting therapies such as anti-CD30, anti-CD19 and anti-CD20 can have limited efficacy due to tumor cell heterogeneity and genomic instability of tumor cells. In addition, mechanisms of resistance can arise such as decreased or heterogeneous target expression, target receptor internalization and drug efflux. ECM proteins rarely have mutations in cancer. The ECM is therefore less heterogeneous and more stable. The ECM is also very abundant and accessible, and fibronectin is a major constituent of tumor ECM. The proteomic data also identify other tumor and/or metastasis-specific ECM proteins and nanobodies against those could similarly be isolated from the M13 libraries in analogous fashion, thereby broadening the range of available targeting reagents using this approach.
5. Advantages over local therapies. Local therapies such as those used in the treatment of GBMs involve local and direct administration of drug to the tumors. Their key limitation is that they are not efficient at treating widely dispersed metastases. Systemic administration of nanobodies allows them to access all diseased tissues, including both primary tumors and metastatic sites throughout the body and they bind selectively to tumor/disease ECM.
6. The NJB2 nanobody recognizes the EIIIB domain of both mouse and human FN. A significant advantage of the NJB2-based nanobodies of the disclosure is that they recognize both the mouse and human EIIIB domain, allowing validation and pre-clinical studies in immuno-competent rodent models and subsequent translation to clinical testing.

### I. Nanobodies

In one aspect, the present disclosure provides nanobodies that bind specifically to the EIIIB domain of fibronectin. The terms “nanobody”, “VHH”, “VHH antibody fragment” and “single-domain antibody” are used without distinction and denote the variable domain of the single heavy chain of antibodies of the type of those found in camelids, which are naturally devoid of light chains. In the absence of a light chain, the nanobodies each have three complementarity determining regions (CDRs), denoted CDR1, CDR2 and CDR3 respectively. The nanobodies according to the invention can be camel, dromedary, llama or alpaca nanobodies. Nanobodies have several advantages over antibody formats for application such as but not limited to, targeted delivery and in vivo diagnostic imaging. Some of the advantages include: (i) small size (2.5 nm dia by 4 nm and Mol wt. of 15 kDa (compared with IgG (150 kDa) allows much faster clearing (renal cut-off for glomerular filtration is 60 kDa) (5); (ii) better tissue penetration compared with full-sized antibodies; (iii) shorter circulatory half-life compared with full-sized antibodies; (iv) the camelid sdAbs (VHH) share a high degree of sequence identity with human VHs and are not known to trigger immunogenic responses in mice; (v) no adverse immunogenic response in human has been reported from nanobodies that are currently in clinical trials; (vi) nanobodies are a renewable resource; (vii) nanobodies are in vivo affinity-matured which is a big advantage over scFvs or man-made scaffolds—as shown in some embodiments, their affinity can be further improved by in vitro selection; (viii) nanobodies are more suitable than scFvs for interacting with grooves on the surface on antigens, such as catalytic sites of enzymes (6); (ix) nanobodies can be humanized if needed (7); and (x) nanobodies are generally stable following addition of cargo such as imaging probes and stable conjugated nanobodies can be generated and purified rapidly and reproducibly.

The present disclosure provides isolated nanobodies. “Isolated nanobodies” as used herein refer to polypeptides that are substantially physically separated from other cellular material (e.g., separated from cells which produce the nanobodies) or from other material that hinders their use either in the diagnostic or therapeutic methods of the invention. In particular, the polypeptides are sufficiently pure and are sufficiently free from other biological constituents of their host cells so as to be useful in, for example, producing pharmaceutical preparations or sequencing. Because isolated nanobodies of the invention may be admixed with a pharmaceutically acceptable carrier in a pharmaceutical preparation, they may comprise only a small percentage by weight of the preparation. The nanobody is nonetheless substantially pure in that it has been substantially separated from the substances with which it may be associated in living systems.

In some embodiments, the nanobodies or other peptides described herein specifically bind to the corresponding target epitope. A nanobody or peptide such as an antibody that “specifically binds” to an epitope is a term well understood in the art. A molecule is said to exhibit “specific binding” if it reacts more frequently, more rapidly, with greater duration and/or with greater affinity with a particular target antigen than it does with alternative targets. A nanobody “specifically binds” to a target antigen or epitope if it binds with greater affinity, avidity, more readily, and/or with greater duration than it binds to other substances. For example, a nanobody that specifically (or preferentially) binds to an antigen or an antigenic epitope therein is a nanobody that binds this target antigen with greater affinity, avidity, more readily, and/or with greater duration than it binds to other antigens or other epitopes in the same antigen. It is also understood with this definition that, for example, a nanobody that specifically binds to a first target antigen may or may not specifically or preferentially bind to a second target antigen. As such, “specific binding” or “preferential binding” does not necessarily require (although it can include) exclusive binding. In some examples, a nanobody that “specifically binds” to a target antigen or an epitope thereof may not bind to other antigens or other epitopes in the same antigen. In some embodiments, a nanobody that specifically binds to the EIIIB domain of fibronectin will bind with lesser affinity (if at all) to other domains of fibronectin or other proteins. Lesser affinity may include at least 10% less, 20% less, 30% less, 40% less, 50% less, 60% less, 70% less, 80% less, 90% less, or 95% less.

In some embodiments, a nanobody as described herein has a suitable binding affinity for the target antigen or antigenic epitopes thereof (e.g., the EIIIB domain of fibronectin). As used herein, “binding affinity” refers to the apparent association constant or KA. The KA is the reciprocal of the dissociation constant (KD). The nanobodies described herein may have a binding affinity (KD) of at least 10⁻⁹, 10⁻¹⁰ M, 10⁻¹¹, 10⁻¹², or lower for the target antigen or antigenic epitope (e.g., the EIIIB domain of fibronectin). An increased binding affinity corresponds to a decreased KD. Higher affinity binding of a nanobody for a first antigen relative to a second antigen can be indicated by a higher KA (or a smaller numerical value KD) for binding the first antigen than the KA (or numerical value KD) for binding the second antigen. In such cases, the nanobody has specificity for the first antigen (e.g., a first protein in a first conformation or mimic thereof) relative to the second antigen (e.g., the same first protein in a second conformation or mimic thereof; or a second protein).

For example, in some embodiments, the anti-FN-EIIIB nanobodies described herein have a higher binding affinity (a higher KA or smaller KD) to the EIIIB domain of fibronectin as compared to the binding affinity to a second antigen or antigenic epitope. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity (or binding specificity) can be determined by a variety of methods including equilibrium dialysis, equilibrium binding, gel filtration, ELISA, surface plasmon resonance, biolayer interferometry or spectroscopy (e.g., using a fluorescence assay).

In some embodiments, the nanobodies have an amino acid sequence that has been “humanized,” i.e., by replacing one or more amino acid residues in the amino acid sequence of the naturally occurring VHH sequence and/or affinity matured VHH sequence (and, in particular, in the framework sequences) by one or more of the amino acid residues that occur at the corresponding position(s) in a VH domain from a conventional four-chain antibody from a human being. Methods for humanization are well known. Humanized nanobodies may have several advantages, such as a reduced immunogenicity, compared to a corresponding naturally occurring VHH domain (or affinity matured VHH domain). “Humanization” can be performed by providing a nucleotide sequence that encodes a naturally occurring VHH domain, and then changing one or more codons in the nucleotide sequence in such a way that the new nucleotide sequence encodes a “humanized” nanobody. This nucleic acid can then be expressed to provide the nanobody. Alternatively, based on the amino acid sequence of a naturally occurring VHH domain the amino acid sequence of the humanized nanobody, can be designed and then synthesized de novo using techniques for peptide synthesis. The skilled artisan may also combine one or more parts of one or more naturally occurring VHH sequences (such as one or more FR sequences or CDR sequences), and/or one or more synthetic or semi-synthetic sequences, in a suitable manner, so as to provide a nanobody or a nucleotide sequence or nucleic acid encoding the same.

A nanobody generally has an amino acid sequence with the structure FR1-CDR1-FR2-CDR2-FR3-CDR3-FR4 in which FR1-FR4 refer to framework regions 1 to 4, respectively, and CDR1 to CDR3 refer to the complementarity determining regions 1 to 3, respectively.

The anti-FN-EIIIB nanobodies of the disclosure are derived from the parental anti-FN-EIIIB nanobody NJB2. In some embodiments, the anti-FN-EIIIB nanobodies of the present disclosure have the following consensus CDR1 sequence: G(S/R/I/N)TFSH(N/S/Y/K)AG(G/S). In some embodiments, the anti-FN-EIIIB nanobodies of the present disclosure have the following consensus CDR2 sequence: (G/R)(I/V)(S/R/G/N)S(D/A/Y)GNI(N/S). In some embodiments, the anti-FN-EIIIB nanobodies of the present disclosure have the following consensus CDR3 sequence: NIRG(S/T)YG(N/S)TYY(S/R)R. The possible amino acid alternatives at each position are indicated in parentheses. In some embodiments, an anti-FN-EIIIB nanobody of the present disclosure comprises a CDR1 comprising G(S/R/I/N)TFSH(N/S/Y/K)AG(G/S). In some embodiments, an anti-FN-EIIIB nanobody of the present disclosure has a CDR2 comprising (G/R)(I/V)(S/R/G/N)S(D/A/Y)GNI(N/S). In some embodiments, an anti-FN-EIIIB nanobody of the present disclosure has a CDR3 comprising NIRG(S/T)YG(N/S)TYY(S/R)R. In some embodiments, an anti-FN-EIIIB nanobody of the disclosure has a) a complementarity-determining region (CDR)1 comprising G(S/R/I/N)TFSH(N/S/Y/K)AG(G/S); b) a CDR2 comprising (G/R)(I/V)(S/R/G/N)S(D/A/Y)GNI(N/S); and c) a CDR3 comprising NIRG(S/T)YG(N/S)TYY(S/R)R. In some embodiments, an anti-FN-EIIIB nanobody of the disclosure does not comprise SEQ ID NO: 1, the sequence of the parental nanobody NJB2.

In some embodiments, an anti-FN-EIIIB nanobody comprises a CDR1 selected from GSTFSHNAGG (SEQ ID NO: 54), GSTFSHSAGG (SEQ ID NO: 57), GSTFSHYAGG (SEQ ID NO: 107), GSTFSHKAGG (SEQ ID NO: 106), GRTFSHNAGG (SEQ ID NO: 105), GSTFSHNAGS (SEQ ID NO: 104), GITFSHNAGG (SEQ ID NO: 103), GRTFSHSAGG (SEQ ID NO: 63), and GNTFSHSAGG (SEQ ID NO: 102). In some embodiments, an anti-FN-EIIIB nanobody comprises a CDR2 selected from GISSDGNIN (SEQ ID NO: 55), GIRSDGNIN (SEQ ID NO: 58), GIGSDGNIN (SEQ ID NO: 59), GISSAGNIN (SEQ ID NO: 60), RISSDGNIN (SEQ ID NO: 108), GINSYGNIN (SEQ ID NO: 109), GIGSAGNIN (SEQ ID NO: 62), GIRSDGNIS (SEQ ID NO: 101), and GVRSDGNIN (SEQ ID NO: 61). In some embodiments, an anti-FN-EIIIB nanobody comprises a CDR3 selected from NIRGSYGNTYYSR (SEQ ID NO: 56), NIRGTYGNTYYSR (SEQ ID NO: 100), NIRGSYGNTYYRR (SEQ ID NO: 99), and NIRGSYGSTYYSR (SEQ ID NO: 98). In some embodiments, an anti-FN-EIIIB nanobody comprises a) a CDR1 selected from GSTFSHNAGG (SEQ ID NO: 54), GSTFSHSAGG (SEQ ID NO: 57), GSTFSHYAGG (SEQ ID NO: 107), GSTFSHKAGG (SEQ ID NO: 106), GRTFSHNAGG (SEQ ID NO: 105), GSTFSHNAGS (SEQ ID NO: 104), GITFSHNAGG (SEQ ID NO: 103), GRTFSHSAGG (SEQ ID NO: 63), and GNTFSHSAGG (SEQ ID NO: 102); b) a CDR2 selected from GISSDGNIN (SEQ ID NO: 55), GIRSDGNIN (SEQ ID NO: 58), GIGSDGNIN (SEQ ID NO: 59), GISSAGNIN (SEQ ID NO: 60), RISSDGNIN (SEQ ID NO: 108), GINSYGNIN (SEQ ID NO: 109), GIGSAGNIN (SEQ ID NO: 62), GIRSDGNIS (SEQ ID NO: 101), and GVRSDGNIN (SEQ ID NO: 61); and c) a CDR3 selected from NIRGSYGNTYYSR (SEQ ID NO: 56), NIRGTYGNTYYSR (SEQ ID NO: 100), NIRGSYGNTYYRR (SEQ ID NO: 99), and NIRGSYGSTYYSR (SEQ ID NO: 98). In some embodiments, an anti-FN-EIIIB nanobody does not comprise SEQ ID NO: 1.

In some embodiments, an anti-FN-EIIIB nanobody comprises a CDR1 comprising GSTFSHSAGG (SEQ ID NO: 57), a CDR2 comprising GIRSDGNIN (SEQ ID NO: 58), and a CDR3 comprising NIRGSYGNTYYSR (SEQ ID NO: 56). In some embodiments, an anti-FN-EIIIB nanobody does not comprise SEQ ID NO: 1.

In some embodiments, an anti-FN-EIIIB nanobody comprises a CDR1 comprising GSTFSHSAGG (SEQ ID NO: 57), a CDR2 comprising GIGSDGNIN (SEQ ID NO: 59), and a CDR3 comprising NIRGSYGNTYYSR (SEQ ID NO: 56). In some embodiments, an anti-FN-EIIIB nanobody does not comprise SEQ ID NO: 1.

In some embodiments, an anti-FN-EIIIB nanobody comprises a CDR1 comprising GSTFSHSAGG (SEQ ID NO: 57), a CDR2 comprising GISSAGNIN (SEQ ID NO: 60), and a CDR3 comprising NIRGSYGNTYYSR (SEQ ID NO: 56). In some embodiments, an anti-FN-EIIIB nanobody does not comprise SEQ ID NO: 1.

In some embodiments, an anti-FN-EIIIB nanobody comprises a CDR1 comprising GSTFSHSAGG (SEQ ID NO: 57), a CDR2 comprising GIGSAGNIN (SEQ ID NO: 62), and a CDR3 comprising NIRGSYGNTYYSR (SEQ ID NO: 56). In some embodiments, an anti-FN-EIIIB nanobody does not comprise SEQ ID NO: 1.

In some embodiments, an anti-FN-EIIIB nanobody comprises a CDR1 comprising GSTFSHSAGG (SEQ ID NO: 57), a CDR2 comprising GVRSDGNIN (SEQ ID NO: 61), and a CDR3 comprising NIRGSYGNTYYSR (SEQ ID NO: 56). In some embodiments, an anti-FN-EIIIB nanobody does not comprise SEQ ID NO: 1.

In some embodiments, an anti-FN-EIIIB nanobody comprises a CDR1 comprising GRTFSHSAGG (SEQ ID NO: 63), a CDR2 comprising GIRSDGNIN (SEQ ID NO: 58), and a CDR3 comprising NIRGSYGNTYYSR (SEQ ID NO: 56). In some embodiments, an anti-FN-EIIIB nanobody does not comprise SEQ ID NO: 1.

In some embodiments, an anti-FN-EIIIB nanobody comprises a CDR1, a CDR2, and a CDR3 of a nanobody comprising the amino acid sequence of any one of SEQ ID Nos. 1-53. In some embodiments, an anti-FN-EIIIB nanobody comprises a CDR1, a CDR2, and a CDR3 of a nanobody comprising the amino acid sequence of any one of SEQ ID Nos. 2-11. In some embodiments, an anti-FN-EIIIB nanobody comprises a CDR1, a CDR2, and a CDR3 of a nanobody comprising the amino acid sequence of SEQ ID NO: 3.

In some embodiments, an anti-FN-EIIIB nanobody comprises a FR1 selected from:

a)
(SEQ ID NO: 64)
QVQLVETGGGLVQAGGSLRLSCAAS;
b)
(SEQ ID NO: 68)
QVQLVGTGGGLVQAGGSLRLSCAAS;
c)
(SEQ ID NO: 85)
RVQLVETGGGLVQAGGSLRLSCAAS;
d)
(SEQ ID NO: 86)
QVRLVETGGGLVQAGGSLRLSCAVS;
e)
(SEQ ID NO: 66)
RVQLVETGGGLVQAGGSLRLSCAVS;
f)
(SEQ ID NO: 71)
QVQLVETGGGLVQAGGSLRLSCAVS;
g)
(SEQ ID NO: 72)
QVQLVETRGGLVQAGGSLRLSCAVS;
h)
(SEQ ID NO: 70)
QVQLVETEGGLVQAGGSLRLSCAAS;
i)
(SEQ ID NO: 87)
RVQLVETEGGLVQAGGSLRLSCAAS;
j)
(SEQ ID NO: 88)
QVRLVETGGGLVQAGGSLRLSCAAS;
k)
(SEQ ID NO: 73)
QVQLVEAGGGLVQAGGSLRLSCAAS;
l)
(SEQ ID NO: 89)
QVQLVETRGGLVQAGGSLRLSCAAS;
m)
(SEQ ID NO: 90)
RVQLVETRGGLVQAGGSLRLSCAAS;
or
n)
(SEQ ID NO: 91)
QVQLVGTGGGLVQAGGSLRLSCAVS.

In some embodiments, an anti-FN-EIIIB nanobody comprises a FR1 selected from:

a)
(SEQ ID NO: 66)
RVQLVETGGGLVQAGGSLRLSCAVS;
b)
(SEQ ID NO: 72)
QVQLVETRGGLVQAGGSLRLSCAVS;
c)
(SEQ ID NO: 71)
QVQLVETGGGLVQAGGSLRLSCAVS;
d)
(SEQ ID NO: 70)
QVQLVETEGGLVQAGGSLRLSCAAS;
e)
(SEQ ID NO: 64)
QVQLVETGGGLVQAGGSLRLSCAAS;
or
f)
(SEQ ID NO: 73)
QVQLVEAGGGLVQAGGSLRLSCAAS.

In some embodiments, an anti-FN-EIIIB nanobody comprises a FR1 comprising RVQLVETGGGLVQAGGSLRLSCAVS (SEQ ID NO: 66). In some embodiments, an anti-FN-EIIIB nanobody does not comprise SEQ ID NO: 1.

In some embodiments, an anti-FN-EIIIB nanobody comprises a FR4 selected from:

a)
(SEQ ID NO: 65)
WGQGTQVTVSS;
b)
(SEQ ID NO: 69)
WGQGIQVTVSS;
c)
(SEQ ID NO: 92)
WSQGTQVTVSS;
d)
(SEQ ID NO: 97)
WGQRTQVTVSS;
e)
(SEQ ID NO: 93)
WGRGAQVTVSS;
f)
(SEQ ID NO: 67)
WGQGAQVTVSS;
g)
(SEQ ID NO: 94)
WGQGTQVTVFS;
h)
(SEQ ID NO: 95)
WGQGTRVTVSS;
or
i)
(SEQ ID NO: 96)
GGQGTQVTVFS.

In some embodiments, an anti-FN-EIIIB nanobody comprises a FR4 selected from: a) WGQGAQVTVSS (SEQ ID NO: 67); b) WGQGTQVTVSS (SEQ ID NO: 65); or c) WGQGIQVTVSS (SEQ ID NO: 69). In some embodiments, an anti-FN-EIIIB nanobody comprises a FR4 comprising WGQGAQVTVSS (SEQ ID NO: 67). In some embodiments, an anti-FN-EIIIB nanobody does not comprise SEQ ID NO: 1.

In some embodiments, an anti-FN-EIIIB nanobody comprises the amino acid sequence of any one of SEQ ID Nos. 2-53. In some embodiments, an anti-FN-EIIIB nanobody consists essentially of the amino acid sequence of any one of SEQ ID Nos. 2-53. In some embodiments, an anti-FN-EIIIB nanobody consists of the amino acid sequence of any one of SEQ ID Nos. 2-53. In some embodiments, an anti-FN-EIIIB nanobody comprises the amino acid sequence of any one of SEQ ID Nos. 2-11. In some embodiments, an anti-FN-EIIIB nanobody consists essentially of the amino acid sequence of any one of SEQ ID Nos. 2-11. In some embodiments, an anti-FN-EIIIB nanobody consists of the amino acid sequence of any one of SEQ ID Nos. 2-11. In some embodiments, an anti-FN-EIIIB nanobody comprises the amino acid sequence of SEQ ID NO: 3. In some embodiments, an anti-FN-EIIIB nanobody consists essentially of the amino acid sequence of SEQ ID NO: 3. In some embodiments, an anti-FN-EIIIB nanobody consists of the amino acid sequence of SEQ ID NO: 3.

In some embodiments, the term nanobody refers to analogs, mutants, variants, alleles, homologs and orthologs of the nanobodies described herein. Generally, in such analogs, one or more amino acid residues may have been replaced, deleted and/or added, compared to the nanobodies disclosed herein. Such substitutions, insertions, deletions or additions may be made in one or more of the framework regions and/or in one or more of the CDRs. In some embodiments, substitutions, insertions, deletions or additions may be made in one or more of the framework regions and/or in one or more of the CDRs of any one of SEQ ID Nos. 1-53. In some embodiments, substitutions, insertions, deletions or additions may be made to a CDR listed in Table 2. In some embodiments, substitutions, insertions, deletions or additions maybe made to a framework region listed in Table 3. In some embodiments, an anti-FN-EIIIB nanobody does not comprise SEQ ID NO: 1.

In some embodiments, an anti-FN-EIIIB nanobody comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared to an amino acid sequence set forth in SEQ ID Nos. 1-53. In some embodiments, an anti-FN-EIIIB nanobody does not comprise SEQ ID NO: 1.

In some embodiments, an anti-FN-EIIIB nanobody comprises up to 5 (e.g., 4, 3, 2, or 1) amino acid residue variations in one or more of the CDR regions of one of the antibodies exemplified herein and binds the same epitope of antigen with substantially similar affinity (e.g., having a KD value in the same order). In some embodiments, an anti-FN-EIIIB nanobody comprises up to 5 (e.g., 4, 3, 2, or 1) amino acid residue variations in one or more of the framework regions of one of the antibodies exemplified herein and binds the same epitope of antigen with substantially similar affinity (e.g., having a KD value in the same order). In one example, the amino acid residue variations are conservative amino acid residue substitutions. As used herein, a “conservative amino acid substitution” refers to an amino acid substitution that does not alter the relative charge or size characteristics of the protein in which the amino acid substitution is made.

“Analogs,” as used herein, are sequences wherein each or any framework region and each or any complementarity-determining region shows at least 80% identity, preferably at least 85% identity, more preferably 90% identity, even more preferably, 95% identity with the corresponding region in the reference sequence (i.e., FR1_analog versus FR1_reference, CDR1_analog versus CDR1_reference, FR2_analog versus FR2_reference, CDR2_analog versus CDR2_reference, FR3_analog versus FR3_reference, CDR3_analog versus CDR3_reference, FR4_analog versus FR4_reference), as measured in a BLASTp alignment.

A substitution may, for example, be a conservative substitution and/or an amino acid residue may be replaced by another amino acid residue that naturally occurs at the same position in another VHH domain. Deletions and/or substitutions may be designed in such a way that one or more sites for post-translational modification (such as one or more glycosylation sites) are removed or to introduce one or more sites for attachment of functional groups, for example, to allow site-specific pegylation.

In some embodiments, an anti-FN-EIIIB nanobody comprise an amino acid sequence that is at least 80%, 81%, 82%, 83%, 84%, 85%, 86%, 87%, 88%, 89%, 90%, 91%, 92%, 93%, 94%, 95%, 96%, 97% 98%, or 99% identical with any one of the amino acid sequences set forth in SEQ ID Nos. 1-53. In some embodiments, an anti-FN-EIIIB nanobody does not comprise SEQ ID NO: 1.

In some embodiments, an anti-FN-EIIIB nanobody of the disclosure comprises a GxS linker. In some embodiments, the GxS linker is at the C-terminus.

The amino acid residues of the nanobody can be modified, i.e., on the protein backbone or on a side chain). Modifications include the introduction of one or more functional groups, residues or moieties into or onto the nanobody. The functional groups may be linked directly to a nanobody or indirectly through a linker or spacer. Methods for increasing the half-life and/or reducing immunogenicity of pharmaceutical proteins involve attaching a polymer, such as poly(ethyleneglycol) (PEG) or derivatives thereof (such as methoxypoly(ethyleneglycol) or mPEG). For example, PEG may be attached to a cysteine residue that naturally occurs in a nanobody or is synthetically added to a nanobody at the terminus or within the nanobody. In some embodiments PEG has a molecular weight of more than 5000 and less than 100,000 or more than 10,000 and less than 200,000. Other modifications include N-linked or O-linked glycosylation, usually as part of co-translational and/or post-translational modification, depending on the host cell used for expressing the nanobody.

Suitable biologically active variants of the nanobodies can be fragments, analogues, and derivatives of the nanobody that specifically bind FN-EIIIB. By “analog” is intended an analog of either the native polypeptide or of a fragment of the native polypeptide, where the analog comprises a native polypeptide sequence and structure having one or more amino acid substitutions, insertions, or deletions. A nanobody fragment is a peptide that is identical to or at least 90% homologous to less than a full length nanobody, referred to herein as a portion of the nanobody. The portion of nanobody or antibody is representative of the full length nanobody or antibody polypeptide. A fragment is representative of the full length nanobody if it includes at least 2 amino acids (contiguous or non-contiguous) of the nanobody and binds to FN-EIIIB. In some embodiments, the portion is less than 90% of the entire native nanobody. In other embodiments, the portion is less than 50%, 45%, 40%, 35%, 30%, 25%, 20%, 15%, 10%, or 5% of the entire native nanobody. By “derivative” is intended any suitable modification of the polypeptide of interest, of a fragment of the polypeptide, or of their respective analogues, such as glycosylation, phosphorylation, polymer conjugation (such as with polyethylene glycol), or other addition of foreign moieties, so long as the desired biological activity of the nanobody or antibody is retained. Methods for making polypeptide fragments, analogues, and derivatives are generally available in the art.

The nanobodies may be obtained from selecting from libraries of such VHH domains, e.g., a phage library or a yeast surface display. A phage library can be created by inserting a library of polynucleotides containing antibody VHH domains from the B-cells of an immunized animal. The diversity of a phagemid library can be manipulated to increase and/or alter the specificities of the polypeptides of the library to produce and subsequently identify additional, desirable, molecular properties and the polynucleotides encoding them. Libraries involve high levels of diversity because it is possible to introduce 1 of 32 different codons in every position and all 20 amino acids. Such a library theoretically grows by 32ⁿ for every n number of residues. Synthesis of oligonucleotides with selected nucleotide “degeneracy” at certain positions is known in that art. Phage display libraries typically have 109 to 1010 “entries.”

Methods of making nanobody libraries generally involve immunization of a camelid (e.g., camel, dromedary, alpaca, vicuna, llama, etc.) with the material to be immunized against. Lymphocytes can then be isolated from blood samples, and lymphocyte RNA can then be used to construct phage display-based nanobody libraries, for example using an M13 phage. The libraries can then be screened for novel nanobodies against a diverse set of ECM and ECM-associated antigens involved in disease. The methods involve harvesting peripheral blood lymphocytes from which nucleic acid can be isolated. The nucleic acid from which the VH domain sequences derive may be total RNA or, more specifically mRNA isolated from the cells within the sample taken from the host. The expression vectors may be any suitable vectors used for library construction, and are preferably phage or phagemid vectors allowing selection of target-specific antibody fragments using phage-display-based selection methods. The antibody sequences isolated from these libraries may then be used as scaffolds for in vitro affinity maturation to further improve binding to the target antigen.

In some embodiments, nanobodies are derived from affinity maturation of an existing nanobody. In some embodiments, affinity maturation involves yeast surface display. Methods of affinity maturation using yeast display are known in the art. See, e.g., Chao G, et al. (2006) Nat Protoc 1(2):755-768.

Further steps may include, for example and without limitation, a step of affinity maturation, a step of expressing and/or modifying the desired amino acid sequence, a step of screening for binding and/or for activity against the desired antigen (ECM epitope), a step of determining the desired amino acid sequence or nucleotide sequence, a step of introducing one or more humanizing substitutions, a step of formatting in a suitable multivalent and/or multispecific format, a step of screening for specific desired biological and/or physiological properties.

In some embodiments, the CDRs or other portions of the nanobodies disclosed herein are incorporated into a structure other than a nanobody. For instance, an antibody (e.g., a humanized antibody) comprising one or more of the CDRs of the nanobodies disclosed herein or analogs thereof may be produced and used according to the invention. The term “antibody” herein is used in the broadest sense and specifically covers intact monoclonal antibodies, multispecific antibodies (e.g., bispecific antibodies) formed from at least two intact antibodies, antibody fragments, so long as they exhibit the desired biological activity, and antibody like molecules such as scFv. A native antibody usually refers to heterotetrameric glycoproteins composed of two identical light (L) chains and two identical heavy (H) chains. Each heavy and light chain has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

Nanobodies can be produced by conventional polypeptide production techniques. For example, they can be synthesized using the well-known solid-phase synthesis method (Merrifield (1962) Proc. Soc. Ex. Boil. 21: 412; Merrifield (1963) J. Am. Chem. Soc. 85: 2149; Tam et al. (1983) J. Am. Chem. Soc. 105: 6442), preferably using a commercially available peptide synthesis instrument (such as the one made by Applied Biosystems, Foster City, Calif.) and by following the manufacturer's instructions.

Alternatively, nanobodies can be synthesized by recombinant DNA techniques well known to those skilled in the art (Maniatis et al. (1982) Molecular Cloning: a laboratory manual, Cold Spring Harbor Laboratories, NY, 51-54 and 412-430). For example, they can be obtained as DNA expression products after incorporation of the DNA sequences encoding the polypeptide of interest into expression vectors and introduction of these vectors into the appropriate prokaryotic or eukaryotic hosts that will express the polypeptide of interest, from which they can then be isolated using techniques well known to those skilled in the art. In some embodiments, polypeptide of interest is linked to a tag that facilitates its purification. Such tags are well known to those skilled in the art and include, but are not limited to, for example, hexahistidine (6His), glutathione S-transferase (GST), the myc tag or influenza virus hemagglutinin (HA). In some embodiments, when a protein is linked to a tag that facilitates its purification, such a protein comprises, between the native sequence and this tag, a sequence which allows enzymatic cleavage between the protein and this tag.

Another aspect of the disclosure relates to a nucleic acid comprising a nucleic sequence encoding a nanobody according to the present disclosure. In some embodiments, a nucleic acid of the disclosure comprises a nucleic acid sequence encoding a nanobody comprising the amino acid sequence of any one of SEQ ID NOS. 2-53. In some embodiments, a nucleic acid of the disclosure comprises a nucleic acid sequence encoding a nanobody that consists essentially of the amino acid sequence of any one of SEQ ID Nos. 2-53. In some embodiments, a nucleic acid of the disclosure comprises a nucleic acid sequence encoding a nanobody that consists of the amino acid sequence of any one of SEQ ID Nos. 2-53. In some embodiments, a nucleic acid of the disclosure comprises a nucleic acid sequence encoding a nanobody that comprises the amino acid sequence of any one of SEQ ID Nos. 2-11. In some embodiments, a nucleic acid of the disclosure comprises a nucleic acid sequence encoding a nanobody that consists essentially of the amino acid sequence of any one of SEQ ID Nos. 2-11. In some embodiments, a nucleic acid of the disclosure comprises a nucleic acid sequence encoding a nanobody that consists of the amino acid sequence of any one of SEQ ID Nos. 2-11. In some embodiments, a nucleic acid of the disclosure comprises a nucleic acid sequence encoding a nanobody that comprises the amino acid sequence of SEQ ID NO: 3. In some embodiments, a nucleic acid of the disclosure comprises a nucleic acid sequence encoding a nanobody that consists essentially of the amino acid sequence of SEQ ID NO: 3. In some embodiments, a nucleic acid of the disclosure comprises a nucleic acid sequence encoding a nanobody that consists of the amino acid sequence of SEQ ID NO: 3.

Typically, said nucleic acid is a DNA or RNA molecule, which can be included in any appropriate vector, such as a plasmid, a cosmid, an episome, an artificial chromosome, a phage or a viral vector. The terms “vector”, “cloning vector” and “expression vector” mean the carrier by which the DNA or RNA sequence can be introduced into the host cell, in such a way as to transform the host and to promote the expression (e.g., transcription and translation) of the sequence introduced. Thus, another aspect of the disclosure relates to a vector comprising a nucleic acid according to the invention.

Such vectors can comprise regulatory elements, such as a promoter, an activator, a terminator, etc., for causing or directing the expression of the polypeptide. Examples of promoters and activators used in expression vectors for animal cells include the SV40 early promoter and activator, the Moloney mouse leukemia virus LTR promoter and activator, the immunoglobulin chain promoter and activator, etc.

Any expression vector for animal cells can be used. Other examples of vectors include replicating plasmids comprising an origin of replication, or integrating plasmids, such as for example pUC, pcDNA, pBR, etc. Other examples of vectors include viral vectors such as adenoviral, retroviral, herpes virus and AAV vectors. Such recombinant viruses can be produced by techniques well known to those skilled in the art, such as by transfection of packaging cells or by transient transfection with helper plasmids or viruses. Typical examples of virus packaging cells include PA317 cells, PsiCRIP cells, GPenv+ cells, 293 cells, etc. Detailed protocols for producing such replication-deficient recombinant viruses can be found for example in applications WO 95/14785, WO 96/22378, U.S. Pat. Nos. 5,882,887, 6,013,516, 4,861,719, 5,278,056 and WO 94/19478.

Another aspect of the present disclosure relates to a cell that has been transfected, transduced or transformed with a nucleic acid and/or a vector according to the invention.

The term “transformation” means the introduction of a “foreign” (i.e. extrinsic or extracellular) gene or DNA or RNA sequence into a host cell, so that the host cell will express the gene or the sequence introduced so as to produce the substance of interest, typically a protein encoded by the gene or the sequence introduced. A host cell which receives and expresses the DNA or RNA introduced has been “transformed”.

The nucleic acids according to the invention can be used to produce a nanobody according to the invention in an appropriate expression system. The term “expression system” means a host cell and a vector compatible under appropriate conditions, e.g., for the expression of a protein encoded by the foreign DNA carried by the vector and introduced into the host cell.

Conventional expression systems include Escherichia coli host cells and plasmid vectors, insect host cells and Baculovirus vectors, and mammalian host cells and their vectors. Other examples of host cells include prokaryotic cells (such as bacteria) and eukaryotic cells (such as yeast cells, mammalian cells, insect cells, plant cells, etc.). Specific examples include Escherichia coli, Kluyveromyces or Saccharomyces yeasts, mammalian cell lines (e.g., Vero cells, CHO cells, 3T3 cells, COS cells, etc.) and also primary or established mammalian cell cultures (e.g., produced from lymphoblasts, fibroblasts, epithelial cells, nerve cells, adipocytes, etc.). Examples also include mouse SP2/0-Ag14 cells (ATCC CRL1581), mouse P3×63-Ag8.653 cells (ATCC CRL1580), CHO cells in which a dihydrofolate reductase gene is defective, rat YB2/3HL.P2.G11.16Ag.20 cells (ATCC CRL1662), etc.

In particular, the invention also relates to a method for producing a nanobody according to the invention, said method comprising the steps of: (i) culturing a cell comprising a nucleic acid or vector of the disclosure under conditions suitable for allowing the expression of said nanobody, and (ii) recovering the nanobody expressed.

The nanobodies according to the invention can be suitably separated from the culture medium by conventional immunoglobulin purification procedures, such as for example protein A-sepharose, hydroxyapatite chromatography, gel electrophoresis, affinity dialysis or chromatography.

### II. Nanobody Conjugates

In some embodiments, an anti-FN-EIIIB nanobody of the disclosure is conjugated to a cargo molecule. The methods of the disclosure involve the derivation, validation and deployment of nanobodies highly specific for an ECM epitope specifically expressed in cancer, metastasis and other disease states. These unique, highly effective nanobodies may be used as conjugated nanobodies in order to enable highly selective means of delivery (with low systemic background) of imaging probes and other cargo molecules including but not limited to drugs, toxins, antibodies, cytokines, siRNAs, shRNAs, nanoparticles, CAR-T cells, CAR-NK cells, CAR-macrophages etc. to an accessible, prevalent, stable extracellular component of diseased tissue. Conjugation techniques and cargo molecules are known in the art, and a brief summary of these is included herein. Alternatively, the nanobodies may be attached to or associated with nanoparticles, which may further comprise other therapeutics, such as, for instance, drugs, toxins, antibodies, cytokines, siRNAs, shRNAs, modified mRNAs, DNA, oligonucleotides, CRISPR Cas9 reagents, imaging probes and other cargo molecules.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin avidin and biotin streptavidin complexation and other affinity interactions. Such means and methods of attachment are well known to those of ordinary skill in the art.

A cargo molecule can be very readily site-specifically attached to the nanobodies for targeted delivery to diseased sites. In some embodiments, the nanobodies may be site-specifically tagged at their N- or C-terminus via sortase-mediated reactions (“sortagging”) to different cargo molecules. This facilitates targeted delivery of any cargo molecule attached to the nanobodies to diseased ECM. ECM remodeling is characteristic of a number of diseases and at least some ECM biomarkers are shared among these diseases. The nanobodies bio-panned from these libraries can therefore find applications across a wide range of diseases that are characterized by ECM remodeling.

In some embodiments, a nanobody conjugate (e.g., a nanobody-cytokine fusion protein) can be produced recombinantly.

The active agent may be conjugated to the N-terminus or the C-terminus or internal amino acids of the nanobody. In some embodiments, a nanobody is directly conjugated to an active agent. A nanobody is directly conjugated to a cargo molecule if the cargo molecule is linked directly (e.g., via a peptide bond) to an amino acid (i.e., N-terminal amino acid, C-terminal amino acid, or internal amino acid) of the nanobody. Alternatively, the nanobody may be indirectly conjugated to a cargo molecule if a linker is used to connect the cargo molecule to the nanobody or the two components may be linked indirectly to one another by linkage to a common carrier molecule.

Thus, linker molecules (“linkers”) may optionally be used to link the nanobody to another molecule. Linkers may be peptides, which consist of one to multiple amino acids, or non-peptide molecules. Examples of peptide linker molecules useful in the invention include glycine-rich peptide linkers (see, e.g., U.S. Pat. No. 5,908,626), wherein more than half of the amino acid residues are glycine. Preferably, such glycine-rich peptide linkers consist of about 20 or fewer amino acids. A specific advantage of the nanobodies is that, during the isolation, subcloning and expression of cloned DNA sequences encoding VHH domain nanobodies, short peptide tags are incorporated that are recognized by sortase enzymes that perform facile conjugation via these tags to other entities (Guimares et al).

A carrier molecule may include, for instance, a PEG or TEG molecule. A PEG or TEG carrier-modified peptide would be referred to as a PEGylated or TEGylated peptide.

Linker molecules may also include non-peptide or partial peptide molecules. For instance, the nanobody may be linked to other molecules using well known cross-linking molecules such as glutaraldehyde or EDC (Pierce, Rockford, Illinois). Bifunctional cross-linking molecules are linker molecules that possess two distinct reactive sites. For example, one of the reactive sites of a bifunctional linker molecule may be reacted with a functional group on a peptide to form a covalent linkage and the other reactive site may be reacted with a functional group on another molecule to form a covalent linkage.

Homobifunctional cross-linker molecules have two reactive sites which are chemically the same. Examples of homobifunctional cross-linker molecules include, without limitation, glutaraldehyde; N,N′-bis(3-maleimido-propionyl-2-hydroxy-1,3-propanediol (a sulfhydryl-specific homobifunctional cross-linker); certain N-succinimide esters (e.g., discuccinimyidyl suberate, dithiobis(succinimidyl propionate), and soluble bis-sulfonic acid and salt thereof.

Preferably, a bifunctional cross-linker molecule is a heterobifunctional linker molecule, meaning that the linker has at least two different reactive sites, each of which can be separately linked to a peptide or other molecule. Use of such heterobifunctional linkers permits chemically separate and stepwise addition (vectorial conjunction) of each of the reactive sites to a selected peptide sequence. Heterobifunctional linker molecules useful in the invention include, without limitation, m-maleimidobenzoyl-N-hydroxysuccinimide ester; m-maleimido-benzoylsulfosuccinimide ester; γ-maleimidobutyric acid N-hydroxysuccinimide ester; and N-succinimidyl 3-(2-pyridyl-dithio)propionate.

The linker may be a “cleavable linker” facilitating release of the cargo molecule in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The carboxyl terminal amino acid residue of the nanobody described herein may also be modified to block or reduce the reactivity of the free terminal carboxylic acid group, e.g., to prevent formation of esters, peptide bonds, and other reactions. Such blocking groups include forming an amide of the carboxylic acid group. Other carboxylic acid groups that may be present in polypeptide may also be blocked, again provided such blocking does not elicit an undesired immune reaction or significantly alter the capacity of the nanobody to specifically function.

Alternatively, the nanobodies may be attached to or associated with nanoparticles, which may further comprise other therapeutics, such as, cargo molecules.

Examples of suitable cargo molecules include, but are not limited to, a molecular imaging probe, a drug, a toxin, an siRNA, an shRNA, an antibody, a cytokine, an enzyme (e.g., ECM modifying enzyme), a CAR-T cell, a radioisotope, or a nanoparticle. In some embodiments, the enzyme is an ECM modifying enzyme. In some embodiments, the enzyme remodels the tumor microenvironment (e.g., glucose oxidase).

In some embodiments, the cargo molecule is a molecular imaging probe. Examples of imaging probes include but are not limited to, radionuclides, fluorophores or other detectable reagents for use in PET, SPECT, NIR, magnetic particle imaging or other imaging modalities, etc.

In some embodiments, the nanobody may be conjugated to a highly radioactive atom. A variety of radioactive isotopes are available for the production of radioconjugated nanobodies. Examples include ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu. When the conjugate is used for detection, it may comprise a radioactive atom for scintigraphic studies, for example ^{99m}Tc or ¹²³I, or a spin label for nuclear magnetic resonance (NMR) imaging (also known as magnetic resonance imaging, MRI), such as ¹²³I, ¹³¹I, ¹¹¹In, ¹⁹F, ¹³C, ¹⁵N, ¹⁷O, Gadolinium, Manganese or Iron. The radio- or other labels may be incorporated in the conjugate in known ways. For example, the binding peptide or polypeptide may be biosynthesized or may be synthesized by chemical amino acid synthesis using suitable amino acid precursors involving, for example, fluorine-19 in place of hydrogen. Labels such as ^{99m}Tc or ¹²³I, ¹⁸⁶Re, ¹⁸⁸Re and ¹¹¹In can be attached via a cysteine residue in the peptide. Yttrium-90 can be attached via a lysine residue. The IODOGEN method (Fraker et al (1978) Biochem. Biophys. Res. Commun. 80: 49-57) can be used to incorporate iodine-123. “Monoclonal Antibodies in Immunoscintigraphy” (Chatal, CRC Press 1989) describes other methods in detail.

In some embodiments, the nanobody is conjugated to a diagnostic and imaging label (generally referred to as in vivo detectable label) such as for example, for magnetic resonance imaging (MRI): Gd(DOTA); for nuclear medicine: 201T1, gamma-emitting radionuclide 99mTc; for positron-emission tomography (PET): positron-emitting isotopes, 18F-fluorodeoxyglucose (18FDG), 18F-fluoride, copper-64, gadodiamide, and radioisotopes of Pb(II) such as 203Pb; 111In.

In some embodiments, the nanobody is conjugated to a drug or a therapeutic. In some embodiments, the drug is an anti-cancer agent. In some embodiments, the cargo molecule is a cytotoxic agent such as a chemotherapeutic agent, toxin (e.g., an enzymatically active toxin of bacterial, fungal, plant or animal origin, or fragments thereof, or a small molecule toxin), or a radioactive isotope (i.e., a radioconjugate) or a CAR-T cell. Other antitumor agents that can be conjugated to the nanobody of the disclosure include BCNU, streptozoicin, vincristine, 5-fluorouracil and other commercially available, FDA approved anti-cancer drugs, the family of agents known collectively LL-E33288 complex described in U.S. Pat. Nos. 5,053,394, 5,770,710, as well as esperamicins (U.S. Pat. No. 5,877,296). Enzymatically active toxins and fragments thereof which can be used in the conjugates include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

Conjugates of the antibody and cargo molecule may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. In some embodiments, the drug is conjugated to the nanobody via a cleavale linker facilitating release of the cargo molecule in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

A CAR-T cell, as used herein, refers to T cells into which a chimeric receptor has been introduced to redirect their specificity towards an antigen of choice. Such receptors comprise an ectodomain that recognizes antigen independent of MHC restriction, in combination with cytoplasmic signaling domains. Many different peptides can be introduced into the T cells as the ectodomain of the chimeric antigen receptors. Examples include a nanobody, a monoclonal antibody, a humanized antibody, a chimeric antibody, a human antibody, or an antibody fragment. In some embodiments, the peptide is a nanobody of the disclosure. In some embodiments, the nanobody is co-expressed in these cells with one or more other CAR receptors.

In addition to conventional CART cells derived from αβT cells, other cells may be used to generate CAR cells which are useful according to the disclosure. For instance, a CAR may be transferred into different cell types, such as γδT cells, natural killer cells, natural killer T cells, and myeloid cells such as macrophage. These cell types possess unique features that may be useful in addressing some of the hurdles of traditional CAR-T-cell therapy.

The nanobodies may also be incorporated into bi-specific T-cell engagers (BiTEs). BiTEs are a class of artificial bi-specific antibodies, linking T cells and tumor cells. They can be used as anti-cancer drugs. The BiTE linkage allows a cytotoxic T cell to recognize a tumor tissue, allowing the T cell to exert its cytotoxic effects without requiring the binding of its specific T cell receptor. The process then parallels the physiological process that occurs when T cells attack tumor cells; the T cells produce proteins, such as perforin and granzymes, which enter the tumor cell and ultimately result in its apoptosis. The entire process occurs independently of the presence of MHC I or other co-stimulatory molecules. BiTEs consist of two single-chain variable fragments (scFvs) or nanobodies. One scFv/nanobody binds to T cells via the CD3 receptor, while the other scFv/nanobody binds to the ECM. There are several clinical trials relating to BiTEs, including MT110, blinaturmomab (MT 103), BAY2010112, MEDI-565, and MEDI-538. Potential clinical applications include treatment of non-Hodgkin's lymphoma, acute lymphoblastic leukemia, gastrointestinal cancers, lung cancers, melanoma, and acute myeloid lymphoma.

In some embodiments, the nanobody is conjugated to a cytokine. Cytokines include but are not limited to IL-2, IL-6, IL-8, IL-10, IL-12, IL-18, TNF, IFN-γ, IFN-β, chemokines, and IFN-α and their mutants. A nanobody-cytokine fusion or conjugate is also referred to herein as an “immunocytokine.”

In some embodiments, the immunocytokine is a nanobody cytokine fusion protein having a nanobody that binds specifically to the EIIIB domain of fibronectin fused to a cytokine. Such fusion proteins can include, for instance, multivalent and multimeric fusion proteins. A multivalent nanobody fusion is a protein comprised of more than 1 copy of the nanobody attached to a therapeutic agent such as a cytokine. A multivalent nanobody may result in an increase in the size or avidity of the therapeutic. For example, a multivalent nanobody structure may have at least two nanobodies fused to the cytokine. In some embodiments, the multivalent fusion protein has 2-3, 2-4, 2-5, 2-6, 2-7, 2-8, 2-9, 2-10, 2-15, 2-20, 2-25, 3-30, 2-35, 2-40, 2-45, 2-45, 2-50, 2-60, 2-70, 2-80, 2-90, 2-100, 3-4, 3-5, 3-6, 3- 7, 3-8, 3-9, 3-10, 3-15, 3-20, 3-25, 3-30, 3-35, 3-40, 3-45, 3-45, 3-50, 3-60, 3-70, 3-80, 3-90, 3-100, 4-5, 4-6, 4-7, 4-8, 4-9, 4-10, 4-15, 4-20, 4-25, 4-30, 4-35, 4-40, 4-45, 4-45, 4-50, 4-60, 4-70, 4-80, 4-90, 4-100, 5-6, 5-7, 5-8, 5-9, 5-10, 5-15, 5-20, 5-25, 5-30, 5-35, 5-40, 5-45, 5-45, 5-50, 5-60, 5-70, 5-80, 5-90, 5-100, 10-100, 10-20, 10-30, 10-40, 10-50, 20-30, 20-40, 20-50, 20-100, 30-40, 30-50, 30-100, 40-50, 40-100, or 50-100 nanobodies fused to the therapeutic agent.

The multivalent fusion protein is comprised of multiple copies of the same nanobody. Such a structure may be depicted as: NB1₍ₙ₌₂₋₅₀₎-cytokine, wherein NB1 refers to a first nanobody e.g., NB1-NB1-cytokine.

In some embodiments, the immunocytokine is a multimeric fusion protein. A multimeric fusion protein is comprised of multiple copies different nanobodies (at least two different nanobodies) fused to a therapeutic agent such as a cytokine. Such a structure may be depicted as: NB1₍ₙ₌₁₋₅₀₎ NB2₍ₙ₌₁₋₅₀₎ NB3₍ₙ₌₀₋₅₀₎ NB4₍ₙ₌₀₋₅₀₎-cytokine, wherein NB1, NB2, NB3, and NB4 refer to a first, second, third, and fourth nanobody e.g., NB1-NB2-cytokine, NB1-NB2-NB3-cytokine, NB1-NB2-NB3-NB4-cytokine. In some embodiments, the multimeric fusion protein has 2-3, 2-4, 2-5, 2-6, 2-7, 2-8, 2-9, 2-10, 2-15, 2-20, 2-25, 3-30, 2-35, 2-40, 2-45, 2-45, 2-50, 2-60, 2-70, 2-80, 2-90, 2-100, 3-4, 3-5, 3-6, 3-7, 3-8, 3-9, 3-10, 3- 15, 3-20, 3-25, 3-30, 3-35, 3-40, 3-45, 3-45, 3-50, 3-60, 3-70, 3-80, 3-90, 3-100, 4-5, 4-6, 4-7, 4-8, 4-9, 4-10, 4-15, 4-20, 4-25, 4-30, 4-35, 4-40, 4-45, 4-45, 4-50, 4-60, 4-70, 4-80, 4-90, 4- 100, 5-6, 5-7, 5-8, 5-9, 5-10, 5-15, 5-20, 5-25, 5-30, 5-35, 5-40, 5-45, 5-45, 5-50, 5-60, 5-70, 5-80, 5-90, 5-100, 10-100, 10-20, 10-30, 10-40, 10-50, 20-30, 20-40, 20-50, 20-100, 30-40, 30-50, 30-100, 40-50, 40-100, or 50-100 different nanobodies fused to the therapeutic agent.

In some embodiments, the fusion protein comprises a nanobody with multiple therapeutic agents such as cytokines attached to either terminal. For instance, the fusion protein might have a structure such as “cytokine 1-NB-cytokine 2” or “NB-cytokine 1-cytokine 2”.

In some embodiments, a nanobody cytokine fusion protein further comprises albumin.

### III. Methods of Use

The anti-FN-EIIIB nanobodies of the disclosure may be used as conjugated nanobodies in order to enable highly selective means of delivery (with low systemic background) of imaging probes and other cargo molecules including but not limited to drugs, toxins, antibodies, cytokines, nanoparticles, CAR-T cells, etc. to an accessible, prevalent, and stable extracellular component of diseased tissue. Alternatively, the nanobodies may be attached to or associated with nanoparticles, which may further comprise other therapeutics, such as, for instance, drugs, toxins, antibodies, cytokines, nucleic acids such as siRNAs, shRNAs, modified mRNAs, DNA, oligonucleotides, CRISPR Cas9 reagents, imaging probes and other cargo molecules.

Thus, in one aspect, the present disclosure provides a method of delivering a cargo molecule to the ECM of diseased tissue in a subject, comprising administering to the subject an effective amount of an anti-FN-EIIIB nanobody of the disclosure conjugated to the cargo molecule.

In some embodiments, the method comprises determining the presence or absence of the EIIIB domain of fibronectin in a subject and determining whether the subject has the disease or condition associated with fibronectin.

In some embodiments, the method comprises measuring the presence or absence of the EIIIB domain of fibronectin in a tissue sample isolated from a subject at a first time point and a second time point and determining the progression of the disease or condition to an advanced stage based on changes in the presence or absence of the EIIIB domain of fibronectin at the first and second time points, wherein i) when the EIIIB domain of fibronectin is present at a higher level in the isolated tissue sample from the second time point, the disease or condition has progressed to an advanced stage, and ii) when the EIIIB domain of fibronectin is present at a lower level in the isolated tissue sample from the second time point, the disease or condition has regressed to a less advanced stage.

In another aspect, the present disclosure provides a method of treating a disease or condition associated with fibronectin, comprising administering to a subject in need thereof an effective amount of an anti-FN-EIIIB nanobody of the disclosure. In some embodiments, the anti-FN-EIIIB nanobody is conjugated to a cargo molecule (e.g., a therapeutic cargo molecule).

Several methods based on application and exploitation of these nanobodies are described herein. Methods for determining the presence of FN-EIIIB from patient samples and thus providing evidence as to whether the subjects have a particular disease and/or the disease's progression or regression are provided. A subject's status can be determined using the anti-FN-EIIIB nanobodies coupled to a cargo molecule using standard immunological methods including immunohistochemistry, immunofluorescence, ELISA, Western blots, protein arrays, noninvasive imaging methods such as Immuno-PET/CT, and similar methods. Use of such methods provides diagnostic, prognostic and monitoring information, allowing for the improved management of patients' care and therapy.

Methods for targeting cargo molecules such as imaging agents (e.g., radionuclides, fluorescent reporters or other detectable reagents for use in PET imaging or other imaging modalities, etc.) specifically to FN-EIIIB within the diseased-state extracellular matrix to detect the location, extent and progression of a disease, e.g., primary tumors and/or metastases are provided.

Methods for targeting active agents such as therapeutic agents (e.g., radionuclides, chemotherapeutic drugs, toxins, cytokines, siRNAs, shRNAs, nanoparticles, CAR-T cells etc.) specifically to FN-EIIIB in order to concentrate such therapeutic agents at diseased tissue, e.g., primary tumors and/or metastases, and thus improve therapeutic index are also provided. Targeting of the therapeutic agents is achieved by attaching them to the anti-FN-EIIIB nanobodies of the disclosure or coformulating them in carriers such as nanoparticles.

In some embodiments, FN-EIIIB may be analyzed in an isolated tissue sample. As used herein, an isolated tissue sample is tissue obtained from a tissue biopsy, a surgically resected tumor, or any other tumor mass removed from the body using methods well known to those of ordinary skill in the related medical arts. The tissue may be known to be diseased or suspected of being diseased (i.e., a disease or condition associated with fibronectin), for example, the tissue may be known to be cancerous or suspected of being cancerous. The phrase “suspected of being cancerous” as used herein means a cancer tissue sample believed by one of ordinary skill in the medical arts to contain cancerous cells. Methods for obtaining the sample from a biopsy include gross apportioning of a mass, microdissection, laser-based microdissection, or other art-known cell-separation methods. The tissue may also be a histological section.

Because of the variability of the cell types in diseased-tissue biopsy material, and the variability in sensitivity of the predictive methods used, the sample size required for analysis may be 5 mg, 10 mg, 15 mg, 25 mg, 30 mg, or 50 mg or greater of tissue. Alternatively, it may be portions or sections of biopsy-sized tissue samples. The appropriate sample size may be determined based on the cellular composition and condition of the biopsy and the standard preparative steps for this determination and subsequent isolation of the proteins for use in the invention are well known to one of ordinary skill in the art.

Alternatively, FN-EIIIB may be analyzed directly in a body. For instance, an anti-FN-EIIIB nanobody may be administered to the subject directly. The nanobody may be labeled, for example with fluorescent, luminescent, NIR, MRI, SPECT, or PET probes in order to assist with visualization of the ECM.

The anti-FN-EIIIB nanobodies of the disclosure may be used to examine changes in the diseased state of a subject (e.g., a disease or condition associated with fibronectin). A diseased state is any physiological condition in a subject caused by under- or overproduction of a biochemical in the subject, invasion of the subject by a foreign organism, i.e., an organism that produces substances that are toxic to the host, or abnormal proliferation of a part of the subject. For example, a diseased state includes, conditions associated with infections, cancers, metastasis, etc. and many of these disease states are associated with excessive production and alteration of ECM, which can be targeted by the nanobodies of this disclosure. Changes in the metastatic state of tumors can be examined over time and/or in response to therapeutic interventions. In order to achieve these methods, tissue samples may be isolated from a subject at different times and/or with or without drug treatments. The different tissue samples can be analyzed for the presence of the FN-EIIIB. Then the differences in FN-EIIIB expression between the two or more samples can be assessed. The differences can be analyzed qualitatively (e.g., by immunohistochemistry, IHC) through assessment of the different proteins present or quantitatively by measuring levels or approximate levels of protein expression (e.g., by ELISA). Alternatively, the methods of the invention may also be performed in vivo without removal of a tissue sample.

The presence of FN-EIIIB in a tissue sample or the level of FN-EIIIB in a tissue sample may be assessed using any known methods in the art. Such methodologies are well known. A method commonly used in clinical and pathological assessment is immunohistochemistry or immunofluorescence, which allows determination of the presence of particular proteins or sets of proteins in a tissue sample. The methods of immunohistochemistry and immunofluorescence are well known and widely practiced. When a quantitative assessment of protein levels is made, the levels of protein may be compared with either a reference or threshold amount or with amounts found in other samples. For instance, if the presence or levels of proteins are measured in a primary tumor and its metastasis or a corresponding normal tissue, those can be compared to provide a relative assessment of the progression of the tumor or its metastases. Alternatively, the levels may be compared with an amount that is known (or is shown) to be an amount above or below which a tumor normally expresses the protein. The value that is used in the comparison is referred to as the reference or threshold level.

The actual numbers in the particular determination of threshold values may vary for different diseases or conditions (e.g., different tumors) or under different circumstances, such as the conditions of the assay to determine expression. However, the skilled artisan would be able to identify the correct threshold values based on the circumstances. For example, threshold values could easily be generated using normal (e.g., non-cancerous) tissue under similar circumstances.

The reference sample can be any of a variety of biological samples against which a diagnostic assessment may be made. Examples of reference samples include biological samples from control populations or control samples. Reference samples may be generated through manufacture to be supplied for testing in parallel with the test samples, e.g., reference sample may be supplied in diagnostic kits. Appropriate reference samples will be apparent to the skilled artisan.

In other embodiments, the expression level of the protein in the test sample may be determined based on a direct comparison to a reference level in absolute values. For instance, at least 10%, at least 20%, at least 50%, at least 100%, at least 200%, at least 500%, at least 1000% or more higher than the expression level of the protein in the reference sample. In other embodiments, the expression level of the protein in the test sample is at least 10%, at least 20%, at least 50%, at least 100%, at least 200%, at least 500%, at least 1000% or more lower than the expression level of the protein in the reference sample. In further embodiments, the expression levels of FN-EIIIB in a subject are determined at a first time point and at a later second time point. Expression levels of FN-EIIB may additionally be measured at further subsequent times, so that the total number of measurements may be 3, 4, 5, 6, 7, 8, 9, 10, or more. The separation in time between any two measurements may be a matter of days, or it may be longer, e.g., weeks, months, or years. For example, the time between obtaining samples is 6 months, 8 months, 10 months, 1 year, 1.5 years, 2 years, 2.5 years, 3 years, 3.5 years, 4 years, 4.5 years, or 5 years. The time between obtaining a first (or earlier) sample and obtaining a subsequent or successive sample the subject may be a time sufficient for a change disease status to occur. The progression or regression of a disease may be determined by comparing the FN-EIIIB expression levels in isolated tissue samples between two or more time points. For example, changes in the presence or absence of FN-EIIIB or their expression levels between successive time points may indicate the progression of a disease, such as to an advanced stage of the disease. In particular, higher expression levels and/or the presence of FN-EIIIB in a sample taken at a later time point may indicate that a disease has progressed to an advanced stage. In some instances, the disease may be cancer, and higher expression levels or wider distribution of FN-EIIB associated with a diseased state in a sample taken at a later time point may indicate that metastatic cancer has progressed. Conversely, if an isolated tissue sample from a second time point has lower expression levels of FN-EIIIB compared to an isolated tissue sample from the first time point, the disease has regressed to a less advanced stage. For example, a successive lower expression level measurement could indicate that a cancer has regressed to a less metastatic state.

The diseased state may be any non-physiological state associated with fibronectin, such as cancer, cardiovascular disease (e.g., atherosclerosis, myocardial infarction), an inflammatory disorder, fibrosis, or a wound. In some embodiments, the cancer is metastatic cancer.

The presence/absence or levels of proteins or markers may be determined using any of a number of techniques available to the person of ordinary skill in the art for protein analysis, e.g., direct physical measurements (e.g., mass spectrometry), or binding assays (e.g., immunohistochemistry, immunoassays, agglutination assays, and immunochromatographic assays) etc. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding domains through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

Binding assays for measuring protein epitope levels may use solid phase or homogenous formats. Suitable assay methods include sandwich or competitive binding assays. Examples of sandwich immunoassays are described in U.S. Pat. No. 4,168,146 to Grubb et al. and U.S. Pat. No. 4,366,241 to Tom et al., both of which are incorporated herein by reference. Examples of competitive immunoassays include those disclosed in U.S. Pat. No. 4,235,601 to Deutsch et al., U.S. Pat. No. 4,442,204 to Liotta, and U.S. Pat. No. 5,208,535 to Buechler et al., all of which are incorporated herein by reference.

Multiple ECM epitopes may be measured using a multiplexed assay format, e.g., multiplexing through the use of nanobody arrays, multiplexing using spectral discrimination of labels, multiplexing by flow cytometric analysis of binding assays carried out on particles.

Detection of a protein epitope in a test sample involves routine methods. The skilled artisan can detect the presence or absence of a protein using well known methods. Such methods include diverse immunoassays. In general, immunoassays involve the binding of antibodies or similar probes to proteins in a sample such a histological section or binding of proteins in a sample to a solid phase support such as a plastic surface. Detectable antibodies (or nanobodies) are added which selectively bind to the protein of interest. Detection of the antibody (or nanobody) indicates the presence of the protein. The detectable antibody (or nanobody) may be a labeled or an unlabeled antibody. Unlabeled antibody (or nanobody) may be detected using a second, labeled antibody that specifically binds to the first antibody (or nanobody) or a second, unlabeled antibody which can be detected using labeled protein A, a protein that complexes with antibodies. Various immunoassay procedures are described in Immunoassays for the 80's, A. Voller et al., Eds., University Park, 1981, which is incorporated herein by reference.

Simple immunoassays such as a dot blot and a Western blot involve the use of a solid phase support which is contacted with a test sample. Any proteins present in the test sample bind the solid phase support and can be detected by a specific, detectable antibody preparation. The intensity of the signal can be measured to obtain a quantitative readout, such as with an ELISA. Other more complex immunoassays include forward assays for the detection of a protein in which a first anti-protein antibody bound to a solid phase support is contacted with the test sample. After a suitable incubation period, the solid phase support is washed to remove unbound protein. A second, distinct anti-protein antibody is then added which is specific for a portion of the specific protein not recognized by the first antibody. The second antibody is preferably detectable. After a second incubation period to permit the detectable antibody to complex with the specific protein bound to the solid phase support through the first antibody, the solid phase support is washed a second time to remove the unbound detectable antibody. Alternatively, in a forward sandwich assay a third detectable antibody, which binds the second antibody is added to the system. Other types of immunometric assays include simultaneous and reverse assays. A simultaneous assay involves a single incubation step wherein the first antibody bound to the solid phase support, the second, detectable antibody and the test sample are added at the same time. After the incubation is completed, the solid phase support is washed to remove unbound proteins. The presence of detectable antibody associated with the solid support is then determined as it would be in a conventional assays. A reverse assay involves the stepwise addition of a solution of detectable antibody to the test sample followed by an incubation period and the addition of antibody bound to a solid phase support after an additional incubation period. The solid phase support is washed in conventional fashion to remove unbound protein/antibody complexes and unreacted detectable antibody.

A number of methods are well known for the detection and quantification of antibodies (or nanobodies). For instance, antibodies can be detectably labeled by linking the antibodies to an enzyme and subsequently using the antibodies in an enzyme immunoassay (EIA) or enzyme-linked immunosorbent assay (ELISA), such as a capture ELISA. The enzyme, when subsequently exposed to its substrate, reacts with the substrate and generates a chemical moiety which can be detected, for example, by spectrophotometric, fluorometric or visual means. Enzymes which can be used to detectably label antibodies include, but are not limited to malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

The methods of the disclosure include methods for in vivo imaging using nanobodies having detectable labels. For instance, the in vivo imaging methods may include PET imaging. PET imaging technologies enable high quality visualization of physiological processes at the molecular level in real time. PET is therefore highly useful in clinical diagnostics and drug development. A number of technical improvements in PET technology have been developed including in the field of tracer development, both progress in labelling strategies and an intelligent design of selective molecular probes with the capability to visualize molecular targets involved in physiological and pathophysiological processes, however, early detection of some tumors and micrometastases remains a challenge.

A detectable label is a moiety, the presence of which can be ascertained directly or indirectly. Generally, detection of the label involves an emission of energy by the label. The label can be detected directly by its ability to emit and/or absorb photons or other atomic particles of a particular wavelength (e.g., radioactivity, luminescence, optical or electron density, etc.). A label can be detected indirectly by its ability to bind, recruit and, in some cases, cleave another moiety which itself may emit or absorb light of a particular wavelength (e.g., epitope tag such as the FLAG epitope, enzyme tag such as horseradish peroxidase, etc.). An example of indirect detection is the use of a first enzyme label which cleaves a substrate into visible products. The label may be of a chemical, peptide or nucleic acid molecule nature although it is not so limited. Labels include any known labels that can be used with imaging techniques, such as PET isotopes, scintigraphy, NMR, etc. Other detectable labels include radioactive isotopes such as P32 or H3, luminescent markers such as fluorochromes, optical or electron density markers, etc., or epitope tags such as the FLAG epitope or the HA epitope, biotin, avidin, and enzyme tags such as horseradish peroxidase, *-galactosidase, as well as nanoparticles, etc. The label may be bound to a reagent during or following its synthesis. There are many different labels and methods of labeling known to those of ordinary skill in the art. Examples of the types of labels that can be used in the present invention include enzymes, radioisotopes, fluorescent compounds, colloidal metals, chemiluminescent compounds, and bioluminescent compounds. Those of ordinary skill in the art will know of other suitable labels for the reagents described herein, or will be able to ascertain such, using routine experimentation. Furthermore, the coupling or conjugation of these labels to the reagents of the invention can be performed using standard techniques common to those of ordinary skill in the art. Therapeutic agents can also be delivered singly and in combinations by nanoparticles of various types and these could be targeted to disease-specific ECM by conjugation with nanoparticles allowing enrichment in the disease site.

Another labeling technique which may result in greater sensitivity consists of coupling the molecules described herein to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

Conjugation of the nanobodies described herein to a detectable label facilitates, among other things, the use of such agents in diagnostic assays. Another category of detectable labels includes diagnostic and imaging labels (generally referred to as in vivo detectable labels) such as for example, for magnetic resonance imaging (MRI): Gd(DOTA); for nuclear medicine: 201T1, gamma-emitting radionuclide 99mTc; for positron-emission tomography (PET): positron-emitting isotopes, 18F-fluorodeoxyglucose (18FDG), 18F-fluoride, copper-64, gadodiamide, and radioisotopes of Pb(II) such as 203Pb; 111In.

The cargo molecule may be a detectable label as described above. Such molecules are useful in vitro or in vivo for detecting and characterizing tumor cells.

In some embodiments, an anti-FN-EIIIB nanobody may be conjugated to a cargo molecule that is a drug or therapeutic. In some embodiments, the anti-FN-EIIIB nanobody is conjugated to an anti-cancer drug. Such compounds may be used as therapeutic conjugates to treat diseases and tumors.

The therapeutic conjugates include an anti-FN-EIIIB nanobody conjugated to a cytotoxic agent such as a chemotherapeutic agent, toxin (e.g., an enzymatically active toxin of bacterial, fungal, plant or animal origin, or fragments thereof, or a small molecule toxin), or a radioactive isotope (i.e., a radioconjugate) or a CAR-T cell. Other antitumor agents that can be conjugated to the anti-FN-EIIIB nanobody include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296). Enzymatically active toxins and fragments thereof which can be used in the conjugates include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

For selective destruction of the cell, the nanobody may be conjugated a highly radioactive atom. A variety of radioactive isotopes are available for the production of radioconjugated antibodies. Examples include 211At, 131I, 125I, 90Y, 186Re, 188Re, 153Sm, 212Bi, 32P, 212Pb and radioactive isotopes of Lu. When the conjugate is used for detection, it may comprise a radioactive atom for scintigraphic studies, for example 99mTc or 123I, or a spin label for nuclear magnetic resonance (NMR) imaging (also known as magnetic resonance imaging, mri), such as 123I, 131I, 111In, 19F, 13C, 15N, 17O, Gadolinium, Manganese or Iron. The radio- or other labels may be incorporated in the conjugate in known ways. For example, the binding peptide or polypeptide may be biosynthesized or may be synthesized by chemical amino acid synthesis using suitable amino acid precursors involving, for example, fluorine-19 in place of hydrogen. Labels such as 99m Tc or 123I, 186Re, 188Re and 111In can be attached via a cysteine residue in the peptide. Yttrium-90 can be attached via a lysine residue. The IODOGEN method (Fraker et al (1978) Biochem. Biophys. Res. Commun. 80: 49-57) can be used to incorporate iodine-123. “Monoclonal Antibodies in Immunoscintigraphy” (Chatal, CRC Press 1989) describes other methods in detail.

In other aspects, the selective destruction of the cells may be mediated by CAR-T cells with a chimeric antigen receptor having an ectodomain comprising an anti-FN-EIIIB of the disclosure.

Additionally, nanobody cytokine conjugates can be used therapeutically. Anti-FN-EIIIB may be used to deliver cytokines which can have a number of functions including enhancing an immune response to the diseased (e.g., a disease associated with fibronectin) tissue (e.g., cancerous tissue). Cytokines include but are not limited to IL-2, IL-6, IL-8, IL-10, IL-12, IL-18, TNF, IFN-γ, IFN-β, chemokines, and IFN-α.

In one aspect, the invention provides methods for the treatment of a disease or condition associated with fibronectin (e.g., a disease associated with the expression of FN-EIIIB). Examples of disorders or diseases associated with fibronecting epitopes include, but are not limited to, cancer, fibrosis, atheromas, inflammatory disorders, cardiovascular diseases, and aneurysms.

In one aspect, the disclosure provides methods for the treatment of cancer. The terms “tumor”, “cancer”, “cancerous tissue” and “carcinoma” are used interchangeably herein, and each, refers to an uncontrolled growth of cells which interferes with the normal functioning of the bodily organs and systems. Cancers, including those cancers which migrate from their original location and seed vital organs, can eventually lead to the death of the subject through the functional deterioration of the affected organs. Cancers can be classified into a variety of categories including, carcinomas, sarcomas and hematopoietic cancers. Carcinomas are malignant cancers that arise from epithelial cells and include adenocarcinoma and squamous cell carcinoma.

As used herein, a subject is a human, non-human primate, cow, horse, pig, sheep, goat, dog, cat, or rodent. In all embodiments human subjects are preferred. In some embodiments, the subject is a human either suspected of having the cancer, or having been diagnosed with cancer. Methods for identifying subjects suspected of having a disease or condition associated with fibronectin (e.g., cancer) may include physical examination, subject's family medical history, subject's medical history, biopsy, or a number of imaging technologies such as ultrasonography, computed tomography, magnetic resonance imaging, magnetic resonance spectroscopy, or positron emission tomography. Diagnostic methods for a disease or condition associated with fibronectin (e.g., cancer) and the clinical delineation of diagnoses are well known to those of skill in the medical arts.

Cancer therapies and their dosages, routes of administration and recommended usage are known in the art. In some embodiments, the therapeutic compounds of the invention are formulated into a pharmaceutical composition that further comprises one or more additional anticancer agents.

The nanobodies of the invention are administered to the subject in an effective amount for treating the subject. An “effective amount”, for instance, is an amount necessary or sufficient to realize a desired biologic effect. For instance, an effective amount is that amount sufficient to prevent or inhibit cancer cell growth or proliferation or alternatively an amount sufficient to induce apoptosis of a cancer cell or induce tumor regression. In some embodiments, the effective amount is that amount useful for reducing the development of metastatic cancers.

The effective amount of a compound of the invention in the treatment of a subject may vary depending upon the specific compound used, the mode of delivery of the compound, and whether it is used alone or in combination. The effective amount for any particular application can also vary depending on such factors as the type and/or degree of cancer in a subject, the particular compound being administered for treatment, the size of the subject, or the severity of the disorder. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity in and of itself and yet is entirely effective to treat the particular subject. Toxicity and efficacy of the protocols of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects. The data obtained from the cell culture assays, animal studies and human studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The term “treat”, “treated”, or “treating” refers to an amelioration of the symptoms associated with a pathological condition afflicting a subject, where amelioration is used in a broad sense to refer to at least a reduction in the magnitude of a parameter, e.g. symptom, associated with the pathological condition being treated, such as a disease or condition associated with fibronectin. In some embodiments, preventing the disease from becoming worse, or slowing the progression of the disease compared to in the absence of the therapy are also encompassed. As such, treatment also includes situations where the pathological condition, or at least symptoms and/or secondary effects associated therewith, are completely inhibited, e.g., prevented from happening, or stopped, e.g., terminated, such that subject no longer suffers from the pathological condition, or at least the symptoms that characterize the pathological condition.

The diagnostic and therapeutic compositions described herein can be administered in combination with other therapeutic agents and such administration may be simultaneous or sequential. When the other therapeutic agents are administered simultaneously, they can be administered in the same or separate formulations, but are administered at the same time. The administration of the other therapeutic agent, including chemotherapeutics can also be temporally separated, meaning that the therapeutic agents are administered at a different time, either before or after, the administration of the therapeutics described herein. The separation in time between the administration of these compounds may be a matter of minutes or it may be longer. When used in combination with the therapies of the disclosure, the dosages of known therapies may be reduced in some instances, to avoid side effects.

In some embodiments, the methods of the disclosure comprise administering another cancer treatment (e.g., radiation therapy, chemotherapy or surgery) to a subject. Examples of conventional cancer therapies include treatment of the cancer with agents such as All-trans retinoic acid, Actinomycin D, Adriamycin, anastrozole, Azacitidine, Azathioprine, Alkeran, Ara-C, Arsenic Trioxide (Trisenox), BiCNU Bleomycin, Busulfan, CCNU, Carboplatin, Capecitabine, Cisplatin, Chlorambucil, Cyclophosphamide, Cytarabine, Cytoxan, DTIC, Daunorubicin, Docetaxel, Doxifluridine, Doxorubicin, 5-flurouracil, Epirubicin, Epothilone, Etoposide, exemestane, Erlotinib, Fludarabine, Fluorouracil, Gemcitabine, Hydroxyurea, Herceptin, Hydrea, Ifosfamide, Irinotecan, Idarubicin, Imatinib, letrozole, Lapatinib, Leustatin, 6-MP, Mithramycin, Mitomycin, Mitoxantrone, Mechlorethamine, megestrol, Mercaptopurine, Methotrexate, Mitoxantrone, Navelbine, Nitrogen Mustard, Oxaliplatin, Paclitaxel, pamidronate disodium, Pemetrexed, Rituxan, 6-TG, Taxol, Topotecan, tamoxifen, taxotere, Teniposide, Tioguanine, toremifene, trimetrexate, trastuzumab, Valrubicin, Vinblastine, Vincristine, Vindesine, Vinorelbine, Velban, VP-16, and Xeloda. Multiple doses of the molecules of the disclosure are also contemplated. In some instances, when the molecules of the disclosure are administered with another therapeutic, for instance, a chemotherapeutic agent a sub-therapeutic dosage of either or both of the molecules may be used. A “sub-therapeutic dose” as used herein refers to a dosage which is less than that dosage which would produce a therapeutic result in the subject if administered in the absence of the other agent.

Pharmaceutical compositions of the present disclosure comprise an effective amount of an anti-FN-EIIIB nanobody of the disclosure, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The compounds may be sterile or non-sterile.

The compositions described herein may comprise different types of carriers depending on whether it is to be administered in solid, liquid or aerosol form, and whether it need to be sterile for such routes of administration as injection.

The composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

A nanobody of the disclosure may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In some embodiments, the compositions include include isotonic agents, such as, for example, sugars, sodium chloride or combinations thereof.

In some embodiments, a nanobody of the disclosure is administered in a pharmaceutically acceptable solution, which may routinely contain pharmaceutically acceptable concentrations of salt, buffering agents, preservatives, compatible carriers, adjuvants, and optionally other therapeutic ingredients. Pharmaceutically acceptable carriers for nucleic acids, small molecules, peptides, monoclonal antibodies, and antibody fragments are well-known to those of ordinary skill in the art. As used herein, a pharmaceutically acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts. The nanobodies of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The disclosure also embraces pharmaceutical compositions which are formulated for local administration, such as by implants, including those designed for slow or controlled release.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compositions can be formulated readily by combining the nanobody with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

For administration by inhalation, the compositions for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

Both non-biodegradable and biodegradable polymeric matrices can be used to deliver the compositions of the disclosure to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

The compositions can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference). In some embodiments, the administration is intravenous. In some embodiments, the administration is intratumoral. In some embodiments, the administration is intraperiotoneal. The compositions of the invention may be administered directly to a tissue. Direct tissue administration may be achieved by direct injection.

The compositions may be administered once, or alternatively they may be administered in a plurality of administrations. If administered multiple times, the compositions may be administered via different routes. For example, the first (or the first few) administrations may be made directly into the affected tissue while later administrations may be systemic.

### IV. Kits

The disclosure also provides kits comprising an anti-FN-EIIIB nanobody of the disclosure. The kit may further comprise assay diluents, standards, controls and/or detectable labels. The assay diluents, standards and/or controls may be optimized for a particular sample. One skilled in the art will readily recognize that reagents of the present invention can be readily incorporated into one of the established kit formats which are well known in the art.

Thus, the agents described herein may, in some embodiments, be assembled into pharmaceutical or diagnostic or research kits to facilitate their use in therapeutic, diagnostic or research applications. A kit may include one or more containers housing the components of the disclosure and instructions for use. “Instructions” typically involve written instructions on or associated with packaging of compositions of the invention. Instructions also can include any oral or electronic instructions provided in any manner. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1. NJB2-IL-2 Immunocytokine

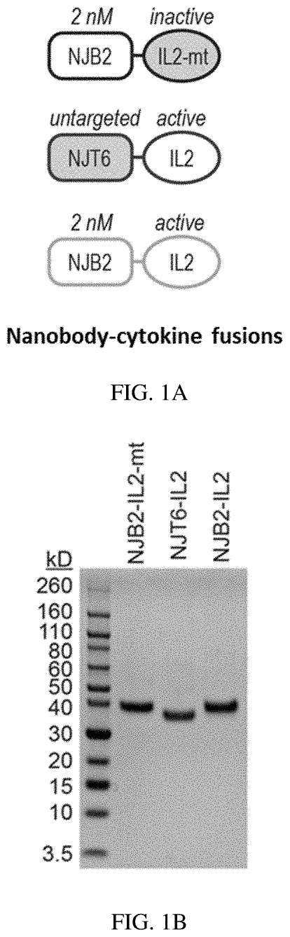

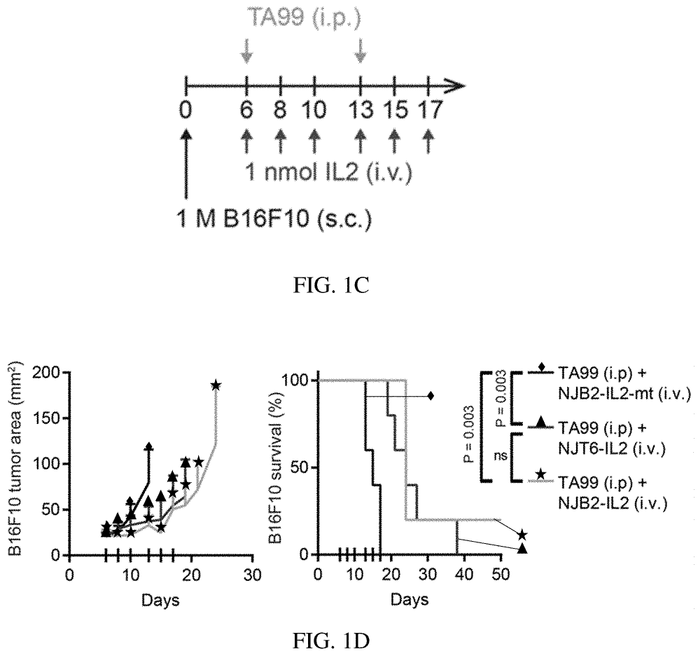

It was tested whether IL2 fused to the FN-EIIIB specific nanobody NJB2 could prolong the survival of disease-bearing mice. Included controls were: (1) a non-targeting but active immunocytokine based on NJT6, which is a nanobody specific to human tenascin C protein, which does not bind to any protein in mice, and (2) an inactive immunocytokine with a “dead” IL2 tagged to NJB2 (FIGS. 1A and 1B). 8 week old mice were inoculated with 1×10⁶ B16F10 cells subcutaneously in the right flank on day 0. Mice were treated on days indicated with 100 μg TA99 (i.p.) and 1 nmol (32 μg) nanobody-IL-2 fusion (i.v.) (FIG. 1C). TA99 is an antibody targeting a surface antigen in melanoma cells. Tumor growth and survival were measured. No significant difference was observed between the NJB2-IL2 and the NJT6-IL2 treated mice (FIG. 1D).

### Example 2. Affinity Maturation of NJB2

When no significant difference was observed between the NJB2-IL2 and NJT6-IL2 treated mice, it was tested whether increasing the affinity of the NJB2 would yield better outcomes.

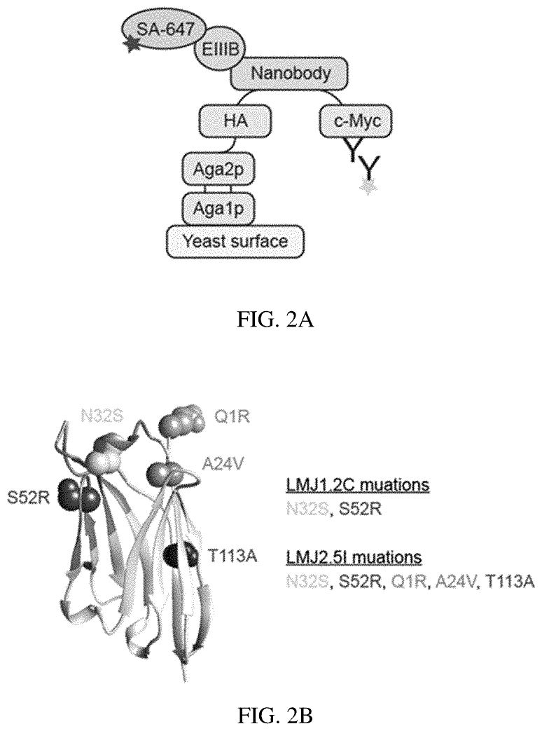

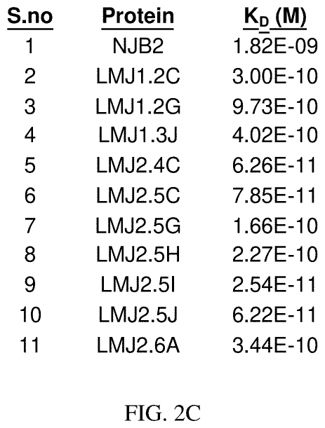

The FN-EIIIB-specific nanobody NJB2 was subjected to random mutagenesis followed by several rounds of affinity maturation using yeast-display methods previously described (2). FIG. 2A shows a schematic of how nanobody libraries were expressed on the surface of yeast as an Aga2 fusion. Nanobody expression is detected by fluorescent antibodies that bind to epitope tags hemagglutinin (HA) or c-Myc. Yeast are selected for enhanced binding to biotinylated FN-EIIIB, as detected by Streptavidin Alexa-Fluor 647 (SA-647).

After two rounds of yeast-surface display, ten clones were selected for affinity characterization using bio-layer interferometry. The second round of yeast-display was done using clones 1.2C, 1.2G and 1.3J from the first round.

The nanobodies were sub-cloned, expressed in E. coli and purified by his-tag purification prior to affinity testing. The KD values are indicated in FIG. 2C. All tested nanobodies had higher affinity than the parent NJB2, with some nanobodies from the second round of yeast display having affinities that were 2 orders of magnitude higher than NJB2.

The sequences of the nanobodies from yeast display are shown in Table 1. In some embodiments, the nanobody comprises a GxS linker at the C-terminus (not shown in Table 1). Diverse mutations both in CDR regions and in framework residues enhanced affinity (Tables 2 and 3). The location of mutations in LMJ1.2C and LMJ2.5I are shown on the predicted structure of NJB2. (FIG. 2B)

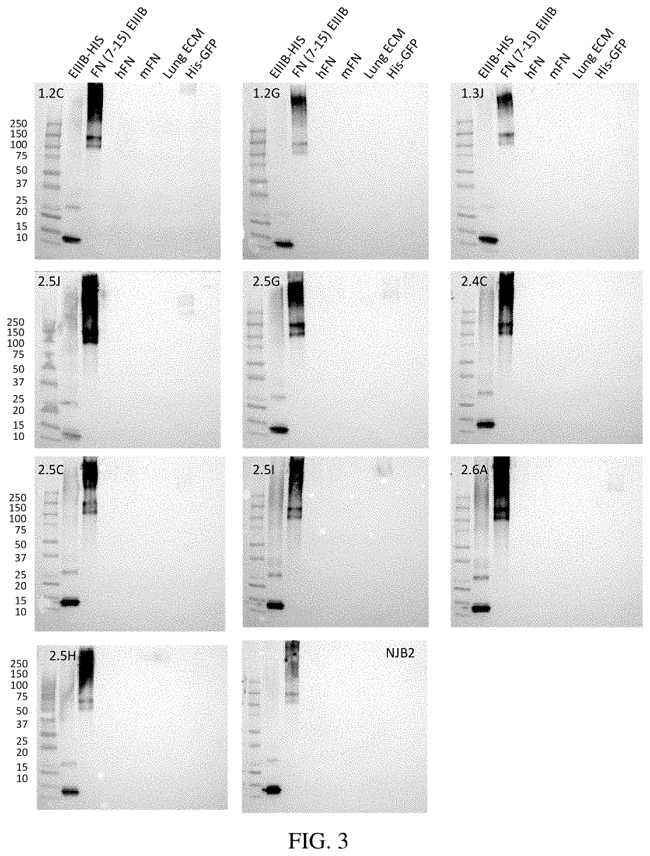

In western blot analysis, the mutants selected by yeast display showed selective binding to FN-EIIIB (FIG. 3). Western blots show that novel nanobodies tagged with biotin detect fragments that contain EIIIB i.e., EIIIB-His and FN (7-15)-EIIIB. They do not bind human or murine plasma fibronectins, which lack EIIIB or normal lung ECM. Thus, the Western blots indicate that the original specificity was retained while the affinity was markedly improved.

TABLE 1
Sequences of nanobodies from yeast display.

| | | SEQ |
|---|---|---|
| Name | Sequence | ID NO: |
| NJB2 | QVQLVETGGGLVQAGGSLRLSCAASGSTFSHNAGGWYRQAPEKQRELVAGIS | 1 |
| (parental | SDGNINYADSVKDRFTISRDNASNTMYLQMNNLKPEDTAVYVCNIRGSYGNT | |
| nanobody) | YYSRWGQGTQVTVSS | |
| LMJ1.2C | QVQLVETGGGLVQAGGSLRLSCAASGSTFSHSAGGWYRQAPEKQRELVAGIR | 2 |
| | SDGNINYADSVKDRFTISRDNASNTMYLQMNNLKPEDTAVYVCNIRGSYGNT | |
| | YYSRWGQGTQVTVSS | |
| LMJ2.5I | RVQLVETGGGLVQAGGSLRLSCAVSGSTFSHSAGGWYRQAPEKQRELVAGIR | 3 |
| | SDGNINYADSVKDRFTISRDNASNTMYLQMNNLKPEDTAVYVCNIRGSYGNT | |
| | YYSRWGQGAQVTVSS | |
| LMJ1.2G | QVQLVETGGGLVQAGGSLRLSCAASGSTFSHSAGGWYRQAPEKQRELVAGIG | 4 |
| | SDGNINYADSVKDRFTISRDNASNTMYLQMNNLKPEDTAVYVCNIRGSYGNT | |
| | YYSRWGQGTQVTVSS | |
| LMJ1.3J | QVQLVGTGGGLVQAGGSLRLSCAASGSTFSHSAGGWYRQAPEKQRELVAGIS | 5 |
| | SAGNINYADSVKDRFTISRDNASNTMYLQMNNLKPEDTAVYVCNIRGSYGNT | |
| | YYSRWGQGTQVTVSS | |
| LMJ2.5C | QVQLVETGGGLVQAGGSLRLSCAASGSTFSHSAGGWYRQAPEKQRELVAGV | 6 |
| | RSDGNINYADSVKDRFTISRDNASNTMYLQMNNLKPEDTAVYVCNIRGSYGN | |
| | TYYSRWGQGIQVTVSS | |
| LMJ2.5G | QVQLVETEGGLVQAGGSLRLSCAASGSTFSHSAGGWYRQAPEKQRELVAGIR | 7 |
| | SDGNINYADSVKDRFTISRDNASNTMYLQMNNLKPEDTAVYVCNIRGSYGNT | |
| | YYSRWGQGTQVTVSS | |
| LMJ2.5H | QVQLVETGGGLVQAGGSLRLSCAVSGSTFSHSAGGWYRQAPEKQRELVAGIR | 8 |
| | SDGNINYADSVKDRFTISRDNASNTMYLQMNNLKPEDTAVYVCNIRGSYGNT | |
| | YYSRWGQGTQVTVSS | |
| LMJ2.5J | QVQLVETRGGLVQAGGSLRLSCAVSGSTFSHSAGGWYRQAPEKQRELVAGIG | 9 |
| | SAGNINYADSVKDRFTISRDNASNTMYLQMNNLKPEDTAVYVCNIRGSYGNT | |
| | YYSRWGQGTQVTVSS | |
| LMJ2.4C | QVQLVEAGGGLVQAGGSLRLSCAASGSTFSHSAGGWYRQAPEKQRELVAGIR | 10 |
| | SDGNINYADSVKDRFTISRDNASNTMYLQMNNLKPEDTAVYVCNIRGSYGNT | |
| | YYSRWGQGIQVTVSS | |
| LMJ2.6A | RVQLVETGGGLVQAGGSLRLSCAVSGRTFSHSAGGWYRQAPEKQRELVAGIR | 11 |
| | SDGNINYADSVKDRFTISRDNASNTMYLQMNNLKPGDTAVYVCNIRGSYGNT | |
| | YYSRWGQGTQVTVSS | |
| LMJ1.2A | RVRLVETGGGLVQAGGSLRLSCAASGSTFSHSAGGWYRQAPEKQRELVAGISS | 12 |
| | DGNINYADSVKDRFTISRDNASNTMYLQMNNLKPEDTAVYVCNIRGSYGNTY | |
| | YSRWGQGTQVTVSS | |
| LMJ1.2B | QVQLVGTGGGLVQAGGSLRLSCAASGSTFSHNAGGWYRQAPEKQRELVAGIS | 14 |
| | SDGNINYADSVKDRFTISRDNASNTMYLQMNNLKPEDAAVYVCNIRGSYGNT | |
| | YYSRWGQGTQVTVSS | |
| LMJ1.2D | QVRLVETGGGLVQAGGSLRLSCAASGSTFSHSAGGWYRQAPEKQRELVAGIS | 15 |
| | SDGNINYADSVKDRFTISRDNASNTMYLQMNNLKPEDTAVYVCNIRGSYGNT | |
| | YYSRWGQGTQVAVSS | |
| LMJ1.2E | QVQLVETRGGLVQAGGSLRLSCAASGSTFSHNAGGWYRQAPEKQRELVAGIS | 16 |
| | SDGNINYADSVKDRFTISRDNASNTMYLQMNNLKPEDTAVYVCNIRGSYGNT | |
| | YYSRWGQGTQVTVSS | |
| LMJ1.2F | QVQLVGTGGGLVQAGGSLRLSCAASGSTFSHNAGGWYRQAPEKQRELVAGIS | 17 |
| | SDGNINYADSVKDRFTISRDNASNTMYLQMNNLKPEDTAVYVCNIRGSYGNT | |
| | YYSRWGQGTRVTVSS | |
| LMJ1.2H | RVQLVETRGGLVQAGGSLRLSCAASGSTFSHNAGGWYRQAPEKQRELVAGIS | 18 |
| | SDGNINYADSVKDRFTISRDNASNTMYLQMNNLKPEDTAVYVCNIRGSYGNT | |
| | YYSRWGQGTQVTVSS | |
| LMJ1.2I | RVQLVETGGGLVQAGGSLRLSCAASGSTFSHNAGGWYRQAPEKQRELVAGIS | 19 |
| | SDGNINYADSVKDRFTISRDNASNTMYLQMNNLKPEDTAVYVCNIRGSYGNT | |
| | YYSRWGQGIQVTVSS | |
| LMJ1.2J | QVQLVGTGGGLVQAGGSLRLSCAASGSTFSHNAGGWYRQAPEKQRELVAGIS | 20 |
| | SDGNINYADSVKDRFTISRDNASNTMYLQMNNLKPEDTAVYVCNIRGSYGNT | |
| | YYSRWGQGTQVTVSS | |
| LMJ1.3A | QVQLVETGGGLVQAGGSLRLSCAASGSTFSHYAGGWYRQAPEKQRELVAGIS | 21 |
| | SDGNINYADSVKDRFTISRDNASNTMYLQMNNLKPGDTAVYVCNIRGSYGNT | |
| | YYSRWGRGTQVTVSS | |
| LMJ1.3B | QVQLVETGGGLVQAGGSLRLSCAASGSTFSHYAGGWYRQAPEKQRELVAGIS | 22 |
| | SDGNINYADSVKDRFTISRDNASNTMYLQMNNLKPEDTAVYICNIRGSYGNTY | |
| | YRRWGQGTQVTVSS | |
| LMJ1.3D | QVQLVETEGGLVQAGGSLRLSCAASGRTFSHNAGGWYRQAPEKQRELVAGIS | 23 |
| | SDGNINYADSVKDRFTISRDNASNTMYLQMNNLKPEDTAVYVCNIRGSYGNT | |
| | YYSRWGQGTQVTVSS | |
| LMJ1.3F | QVRLVETGGGLVQAGGSLRLSCAASGSTFSHSAGGWYRQAPEKQRELVAGIS | 24 |
| | SDGNINYADSVKDRFTISRDNASNTMYLQMNNLKPEDTAVYVCNIRGSYGNT | |
| | YYSRWGQGTQVTVSS | |
| LMJ1.3G | QVQLVETGGGLVQAGGSLRLSCAASGSTFSHKAGGWYRQAPEKQRELVARIS | 25 |
| | SDGNINYADSVKDRFTISRDNASNTMYLQMNNLKPEDTAVYVCNIRGSYGNT | |
| | YYSRWGQGTQVTVSS | |
| LMJ1.3H | QVQLVETGGGLVQAGGSLRLSCAASGSTFSHNAGGWYRQAPEKQRELVAGIS | 26 |
| | SDGNINYADSVKDRFTISRDNASNTMYLQMNNLKPEDTAVYVCNIRGSYGNT | |
| | YYSRWGQRTQVTVSS | |
| LMJ1.4A | QVRLVETGGGLVQAGGSLRLSCAASGSTFSHNAGGWYRQAPEKQRELVAGIN | 27 |
| | SYGNINYADSVKDRFTISRDNASNTMYLQMNNLKPEDTAVYVCNIRGSYGNT | |
| | YYSRWGQGTQVTVSS | |
| LMJ1.4B | QVQLVKTGGGLVQAGGSLRLSCAASGSTFSHNAGGWYRQAPEKQRELVAGIS | 28 |
| | SDGNINYADSVKDRFTISRDNASNTMYLQMNNLKPEDTAVYVCNIRGTYGNT | |
| | YYSRWGQGTQVTVSS | |
| LMJ1.4C | QVQLVGTGGGLVQAGGSLRLSCAVSGSTFSHNAGGWYRQAPEKQRELVAGIS | 29 |
| | SDGNINYADSVKDRFTISRDNASNTMHLQMNNLKPEDTAVYVCNIRGSYGNT | |
| | YYSRWGQGTQVTVSS | |
| LMJ1.4D | QVQLVETRGGLVQAGGSLRLSCAASGITFSHNAGGWYRQAPEKQRELVAGIS | 30 |
| | SDGNINYADSVKDRFTISRDNASNTMYLQMNNLKPEDTAVYVCNIRGSYGNT | |
| | YYSRWGQGTQVTVSS | |
| LMJ1.4E | QVQLVGTGGGLVQAGGSLRLSCAASGSTFSHNAGSWYRQAPEKQRELVAGIR | 31 |
| | SDGNINYADSVKDRFTISRDNASNTMYLQMNNLKPEDTAVYVCNIRGSYGNT | |
| | YYSRWGQGTQVTVSS | |
| LMJ1.4G | RVQLVETGGGLVQAGGSLRLSCAASGSTFSHNAGGWYRQAPEKQRELVAGIS | 32 |
| | SDGNINYADSVKDRFTISRDNASNTMYLQMNNLKPEDTAVYVCNIRGSYGNT | |
| | YYSRWGQRTQVTVSS | |
| LMJ1.4H | RVQLVETGGGLVQAGGSLRLSCAASGSTFSHSAGGWYRQAPEKQRELVAGIS | 33 |
| | SDGNINYADSVKDRFTISRDNASNTMYLQMNNLKPEDTAVYVCNIRGSYGNT | |
| | YYSRWGQGTRVTVSS | |
| LMJ2.3A | QVRLVETGGGLVQAGGSLRLSCAVSGSTFSHSAGGWYRQAPEKQRELVAGIS | 34 |
| | SAGNINYADSVKDRFTISRDNASNTMYLQMNNLKPEDTAVYVCNIRGSYGNT | |
| | YYSRWGQGTQVTVSS | |
| LMJ2.3B | QVQLVGTGGGLVQAGGSLRLSCAASGSTFSHSAGGWYRQAPGKQRELVAGIR | 35 |
| | SDGNINYADSVKDRFTISRDNASNTMYLQMNNLRPEDTAVYVCNIRGSYGNT | |
| | YYSRWGQGTQVTVSS | |
| LMJ2.3C | QVQLVETGGGLVQAGGSLRLSCAASGSTFSHSAGGWYRQAPEKQRELVAGIR | 36 |
| | SDGNINYADSVKDRFTISRDNASNTMYLQMNNLKPEDTAVYVCNIRGSYGST | |
| | YYSRWGQGTQVTVSS | |
| LMJ2.3D | QVQLVETGGGLVQAGGSLRLSCAASGSTFSHSAGGWYRQAPEKQRELVAGIS | 37 |
| | SAGNINYADSVKDRFTISRDNASNTMYLQMNNLKPEDTAVYVCNIRGSYGNT | |
| | YYSRWGQRTQVTVSP | |
| LMJ2.3E | QVQLVETGGGLVQAGGSLRLSCAASGSTFSHSAGGWYRQAPEKQRELVAGIR | 38 |
| | SDGNINYADSVKDRFTISRDNASNTMYLQMDNLKPEDTAVYVCNIRGSYGNT | |
| | YYSRWSQGTQVTVSS | |
| LMJ2.3F | RVQLVETGGGLVQAGGSLRLSCAASGSTFSHSAGGWYRQAPEKQRELVAGIR | 39 |
| | SDGNINYADSVKDRFTISRDNASNTMYLQMNNLKPEDTAVYVCNIRGSYGNT | |
| | YYSRWGQGTQVTVSS | |
| LMJ2.3G | QVQLVGTGGGLVQAGGSLRLSCAASGSTFSHSAGGWYRQAPEKQRELVAGIR | 40 |
| | SDGNINYADSVKDRFTISRDNASNTMYLQMNNLKPEDTAVYVCNIRGSYGNT | |
| | YYSRWGQGTQVTVSS | |
| LMJ2.3I | RVQLVETGGGLVQAGGSLRLSCAASGSTFSHSAGGWYRQAPEKQRELVAGIS | 41 |
| | SAGNINYADSVKDRFTISRDNASNTMYLQMNNLKPEDTAVYVCNIRGSYGNT | |
| | YYSRWGQGIQVTVSS | |
| LMJ2.3J | RVQLVETGGGLVQAGGSLRLSCAASGSTFSHSAGGWYRQAPEKQRELVAGIS | 42 |
| | SAGNINYADSVKDRFTISKDDASNTMYLQMNNLKPEDTAVYVCNIRGSYGNT | |
| | YYSRWGQGTQVTVSS | |
| LMJ2.3H | QVQLVETGGGLVQAGGSLRLSCAASGSTFSHSAGGWYRQAPEKQRELVAGIR | 43 |
| | SDGNINYADSVKDRFTISRDNASNTMYLQMNNLKPEDTAVYVCNIRGSYGNT | |
| | YYSRWGRGAQVTVSS | |
| LMJ2.4D | QVQLVETGGGLVQAGGSLRLSCAVSGSTFSHSAGGWYRQAPEKQRELVAGIR | 44 |
| | SDGNINYADSVKDRFTISRGNASNTMYLQMNNLKPEDTAVYVCNIRGSYGNT | |
| | YYSRWGQGTQVTVSS | |
| LMJ2.5A | QVQLVETEGGLVQAGGSLRLSCAASGSTFSHSAGGWYRQAPEKQRELVAGIR | 7 |
| | SDGNINYADSVKDRFTISRDNASNTMYLQMNNLKPEDTAVYVCNIRGSYGNT | |
| | YYSRWGQGTQVTVSS | |
| LMJ2.5B | RVQLVETEGGLVQAGGSLRLSCAASGSTFSHSAGGWYRQAPEKQRELVAGIG | 45 |
| | SDGNINYADSVKDRFTISRDNASNTMYLQMNNLKPEDTAVYVCNIRGSYGNT | |
| | YYSRWGQGTQVTVSS | |
| LMJ2.5D | RVQLVETGGGLVQAGGSLRLSCAASGSTFSHSAGGWYRQAPEKQRELVAGIR | 46 |
| | SDGNINYADSVKGRFTISRDNASNTMYLQMNNLKPEDTAVYVCNIRGSYGNT | |
| | YYSRWGQGIQVTVSS | |
| LMJ2.5E | QVRLVETGGGLVQAGGSLRLSCAASGSTFSHSAGGWYRQAPEKQRELVVGIR | 47 |
| | SDGNISYADSVKDRFTISRDNASNTMYLQMNNLKPEDTAVYVCNIRGSYGNT | |
| | YYSRWGQGTQVTVSS | |
| LMJ2.5F | QVQLVETGGGLVQAGGSLRLSCAASGSTFSHSAGGWYRQAPEKQRELVAGIR | 48 |
| | SDGNINYADSVKGRFTISRDNASNTMYLQMNNLKPEDTAVYVCNIRGSYGNT | |
| | YYSRWGQRTQVTVSS | |
| LMJ2.5K | RVQLVETGGGLVQAGGSLRLSCAASGNTFSHSAGGWYRQAPEKQRELVAGIR | 49 |
| | SDGNISYADSVKDRFTISRDNASNTMYLQMNNLKPEDTAVYVCNIRGSYGNT | |
| | YYSRWGQGTQVTVSS | |
| LMJ2.5N | QVQLVETGGGLVQAGGSLRLSCAVSGSTFSHSAGGWYRQAPEKQRELVAGIR | 50 |
| | SDGNINYADSVKDRLTISRDNASNTMYLQMNNLKPEDTAVYVCNIRGSYGNT | |
| | YYSRWGQGTQVTVSS | |
| LMJ2.6D | RVQLVETGGGLVQAGGSLRLSCAVSGRTFSHSAGGWYRQAPEKQRELVAGIR | 51 |
| | SDGNINYADSVKDRFTISRDNASNTMYLQMNNLKPGDTGVYVCNIRGSYGNT | |
| | YYSRGGQGTQVTVFS | |
| LMJ1.2G | RVQLVETGGGLVQAGGSLRLSCAASGSTFSHSAGGWYRQAPEKQRELVAGIG | 52 |
| A | SDGNINYADSVKDRFTISRDNASNTMYLQMNNLKPEDTAVYVCNIRGSYGNT | |
| | YYSRWGQGTQVTVSS | |
| LMJ2.6A | RVQLVETGGGLVQAGGSLRLSCAVSGRTFSHSAGGWYRQAPEKQRELVAGIR | 53 |
| A | SDGNINYADSVKDRFTISRDNASNTMYLQMNNLKPGDTAVYVCNIRGSYGNT | |
| | YYSRWGQGTQVTVFS | |

The CDR regions are underlined. The tested clones are shown in bold.

TABLE 2
CDRs of nanobodies tested for affinity and specificity

| Nanobody | CDR1 | CDR2 | CDR3 |
|---|---|---|---|
| NJB2 (parental | GSTFSHNAGG (SEQ | GISSDGNIN (SEQ ID | NIRGSYGNTYYSR (SEQ |
| nanobody) | ID NO: 54) | NO: 55) | ID NO: 56) |
| LMJ1.2C | GSTFSHSAGG (SEQ | GIRSDGNIN (SEQ ID | NIRGSYGNTYYSR (SEQ |
| | ID NO: 57) | NO: 58) | ID NO: 56) |
| LMJ2.5I | GSTFSHSAGG (SEQ | GIRSDGNIN (SEQ ID | NIRGSYGNTYYSR (SEQ |
| | ID NO: 57) | NO: 58) | ID NO: 56) |
| LMJ1.2G | GSTFSHSAGG (SEQ | GIGSDGNIN (SEQ ID | NIRGSYGNTYYSR (SEQ |
| | ID NO: 57) | NO: 59) | ID NO: 56) |
| LMJ1.3J | GSTFSHSAGG (SEQ | GISSAGNIN (SEQ ID | NIRGSYGNTYYSR (SEQ |
| | ID NO: 57) | NO: 60) | ID NO: 56) |
| LMJ2.5C | GSTFSHSAGG (SEQ | GVRSDGNIN (SEQ ID | NIRGSYGNTYYSR (SEQ |
| | ID NO: 57) | NO: 61) | ID NO: 56) |
| LMJ2.5G | GSTFSHSAGG (SEQ | GIRSDGNIN (SEQ ID | NIRGSYGNTYYSR (SEQ |
| | ID NO: 57) | NO: 58) | ID NO: 56) |
| LMJ2.5H | GSTFSHSAGG (SEQ | GIRSDGNIN (SEQ ID | NIRGSYGNTYYSR (SEQ |
| | ID NO: 57) | NO: 58) | ID NO: 56) |
| LMJ2.5J | GSTFSHSAGG (SEQ | GIGSAGNIN (SEQ ID | NIRGSYGNTYYSR (SEQ |
| | ID NO: 57) | NO: 62) | ID NO: 56) |
| LMJ2.4C | GSTFSHSAGG (SEQ | GIRSDGNIN (SEQ ID | NIRGSYGNTYYSR (SEQ |
| | ID NO: 57) | NO: 58) | ID NO: 56) |
| LMJ2.6A | GRTFSHSAGG (SEQ | GIRSDGNIN (SEQ ID | NIRGSYGNTYYSR (SEQ |
| | ID NO: 63) | NO: 58) | ID NO: 56) |

Changes in the CDRs relative to the corresponding CDRs of NJB2 are underlined.

TABLE 3
Frameworks 1 and 4 of nanobodies tested for affinity and specificity

| Nanobody | Framework 1 | Framework 4 |
|---|---|---|
| NJB2 (parental | QVQLVETGGGLVQAGGSLRLSCAAS | WGQGTQVTVSS (SEQ ID NO: |
| nanobody) | (SEQ ID NO: 64) | 65) |
| LMJ1.2C | QVQLVETGGGLVQAGGSLRLSCAAS | WGQGTQVTVSS (SEQ ID NO: |
| | (SEQ ID NO: 64) | 65) |
| LMJ2.5I | RVQLVETGGGLVQAGGSLRLSCAVS | WGQGAQVTVSS (SEQ ID NO: |
| | (SEQ ID NO: 66) | 67) |
| LMJ1.2G | QVQLVETGGGLVQAGGSLRLSCAAS | WGQGTQVTVSS (SEQ ID NO: |
| | (SEQ ID NO: 64) | 65) |
| LMJ1.3J | QVQLVGTGGGLVQAGGSLRLSCAAS | WGQGTQVTVSS (SEQ ID NO: |
| | (SEQ ID NO: 68) | 65) |
| LMJ2.5C | QVQLVETGGGLVQAGGSLRLSCAAS | WGQGIQVTVSS (SEQ ID NO: |
| | (SEQ ID NO: 64) | 69) |
| LMJ2.5G | QVQLVETEGGLVQAGGSLRLSCAAS | WGQGTQVTVSS (SEQ ID NO: |
| | (SEQ ID NO: 70) | 65) |
| LMJ2.5H | QVQLVETGGGLVQAGGSLRLSCAVS | WGQGTQVTVSS (SEQ ID NO: |
| | (SEQ ID NO: 71) | 65) |
| LMJ2.5J | QVQLVETRGGLVQAGGSLRLSCAVS | WGQGTQVTVSS (SEQ ID NO: |
| | (SEQ ID NO: 72) | 65) |
| LMJ2.4C | QVQLVEAGGGLVQAGGSLRLSCAAS | WGQGIQVTVSS (SEQ ID NO: |
| | (SEQ ID NO: 73) | 69) |
| LMJ2.6A | RVQLVETGGGLVQAGGSLRLSCAVS | WGQGTQVTVSS (SEQ ID NO: |
| | (SEQ ID NO: 66) | 65) |

Changes in the framework 1 and 4 regions relative to the corresponding framework regions of NJB2 are underlined.

### Example 3. Testing Nanobody-Cytokine Fusions in Tumor Models by Intravenous Administration

The efficacy of nanomolar and picomolar IL-2 immunocytokines specific for FN-EIIIB was tested in the B16F10 and 4T1 tumor models. It was found that nanomolar and picomolar IL-2 immunocytokines specific for FN-EIIIB lead to modest delay of Bl6F10 and 4T1 tumor growth after intravenous administration.

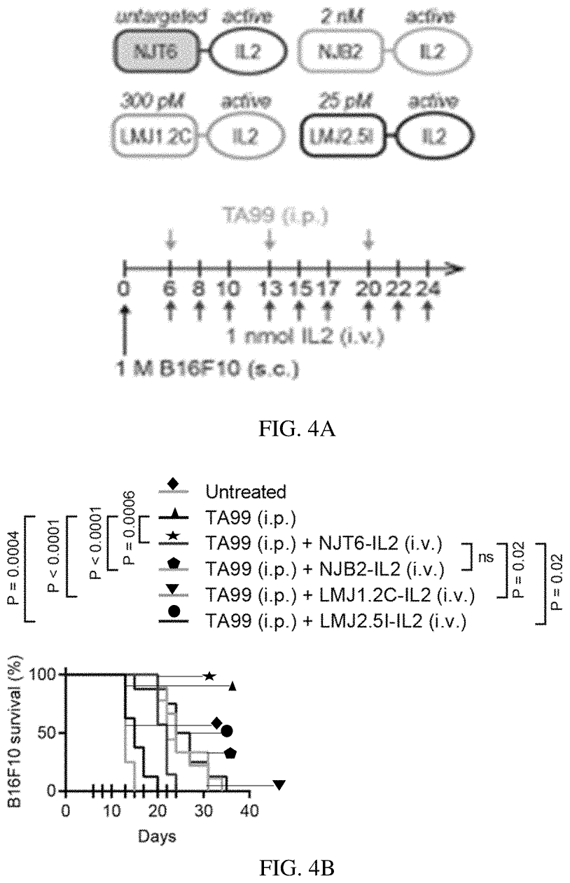

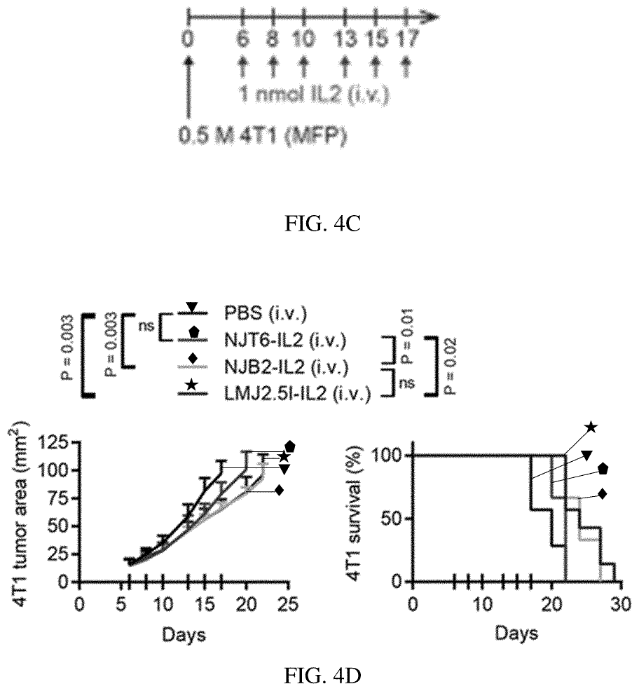

6-12 week-old mice were inoculated with 1×10⁶B16F10 cells subcutaneously (s.c.) in the right flank on day 0. Mice were treated on days indicated with 100 μg TA99 (i.p.) and 1 nmol (32 μg) of the following nanobody IL-2 fusions (i.v.): (1) NJB2-IL2 (based on parental nanobody NJB2), (2) LMJ1.2C-IL2, (3) LMJ2.5I-IL2, and (4) NJT6-IL2 (non-targeting control based on NJT6, a nanobody specific for the human TNC protein) (FIG. 4A). NJB2-IL2 extends survival in disease-bearing mice compared to NJT6-IL2 (non-targeting control) (FIG. 4B). While the extension in the B16F10 model was not statistically significant, the trend is promising.

6-10 week-old mice were inoculated with 0.5×10⁶ 4T1 cells in the mammary fat pad (MFP) on day 0 and treated on days indicated with 1 nmol (32 μg) of the following nanobody IL-2 fusions (i.v.): (1) NJB2-IL2 (based on parental nanobody NJB2), (2) LMJ2.5I-IL2, and (3) NJT6-IL2 (non-targeting control based on NJT6, a nanobody specific for the human TNC protein) (FIG. 4C). Tumor growth and survival were assessed. NJB2-IL2 and LMJ2.5I-IL2 extend survival in disease-bearing mice compared to NJT6-IL2 (non-targeting control) (FIG. 4D). In the 4T1 model this extension was statistically significant.

Sequences of the nanobody-IL2 fusions (Nanobody—GGGGGS—murine IL2—LPETGG—HHHHHH) tested in Examples 3 and 4 are provided below.

NJT6-IL2:
(SEQ ID NO: 74)
QVQLVETGGDLVQPGGSLRLSCAASGLTLDYYAIGWVRQAPGKEREGVS
CITPQDGNTYYDDSVMGRFTILRDNAKNMVYLQMNNLKPEDTAVYFCAA
AGALTLDPSEYEYWGQGTQVTVSSGGGGGSAPTSSSTSSSTAEAQQQQQ
QQQQQQQHLEQLLMDLQELLSRMENYRNLKLPRMLTFKFYLPKQATELK
DLQCLEDELGPLRHVLDLTQSKSFQLEDAENFISNIRVTVVKLKGSDNT
FECQFDDESATVVDFLRRWIAFCQSIISTSPQLPETGGHHHHHH
NJB2-IL2:
(SEQ ID NO: 75)
QVQLVETGGGLVQAGGSLRLSCAASGSTFSHNAGGWYRQAPEKQRELVA
GISSDGNINYADSVKDRFTISRDNASNTMYLQMNNLKPEDTAVYVCNIR
GSYGNTYYSRWGQGTQVTVSSGGGGGSAPTSSSTSSSTAEAQQQQQQQQ
QQQQHLEQLLMDLQELLSRMENYRNLKLPRMLTFKFYLPKQATELKDLQ
CLEDELGPLRHVLDLTQSKSFQLEDAENFISNIRVTVVKLKGSDNTFEC
QFDDESATVVDFLRRWIAFCQSIISTSPQLPETGGHHHHHH
LMJ1.2C-IL2:
(SEQ ID NO: 76)
QVQLVETGGGLVQAGGSLRLSCAASGSTFSHSAGGWYRQAPEKQRELVA
GIRSDGNINYADSVKDRFTISRDNASNTMYLQMNNLKPEDTAVYVCNIR
GSYGNTYYSRWGQGTQVTVSSGGGGGSAPTSSSTSSSTAEAQQQQQQQQ
QQQQHLEQLLMDLQELLSRMENYRNLKLPRMLTFKFYLPKQATELKDLQ
CLEDELGPLRHVLDLTQSKSFQLEDAENFISNIRVTVVKLKGSDNTFEC
QFDDESATVVDFLRRWIAFCQSIISTSPQLPETGGHHHHHH
LMJ2.5I-IL2:
(SEQ ID NO: 77)
RVQLVETGGGLVQAGGSLRLSCAVSGSTFSHSAGGWYRQAPEKQRELVA
GIRSDGNINYADSVKDRFTISRDNASNTMYLQMNNLKPEDTAVYVCNIR
GSYGNTYYSRWGQGAQVTVSSGGGGGSAPTSSSTSSSTAEAQQQQQQQQ
QQQQHLEQLLMDLQELLSRMENYRNLKLPRMLTFKFYLPKQATELKDLQ
CLEDELGPLRHVLDLTQSKSFQLEDAENFISNIRVTVVKLKGSDNTFEC
QFDDESATVVDFLRRWIAFCQSIISTSPQLPETGGHHHHHH.

### Example 4. Testing Nanobody-Cytokine Fusions in Tumor Models by Intratumoral Administration

To avoid challenges/limitations associated with systemic delivery, it was tested whether intra-tumoral delivery circumvents this problem and improves survival.

The efficacy of the nanobody-cytokine fusions was tested in the B16F10 solid tumor model by intratumoral administration. It was found that intratumoral administration of FN-EIIIB specific IL-2 immunocytokines enables high B16F10 cure rate.

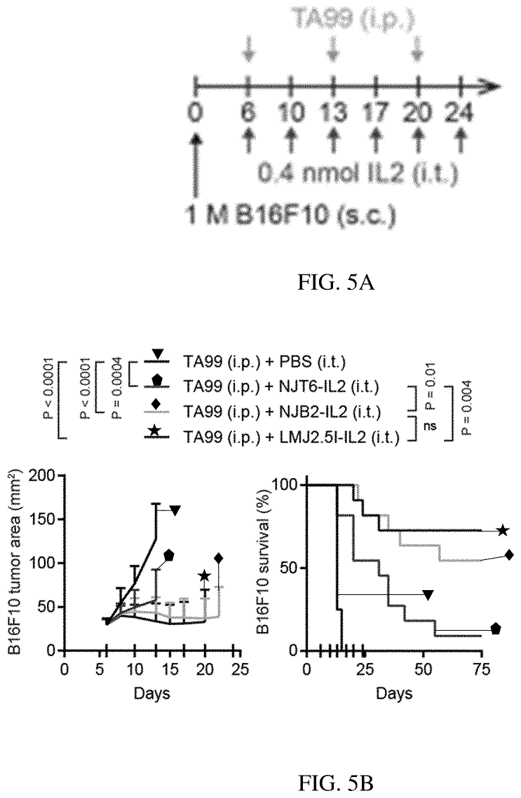

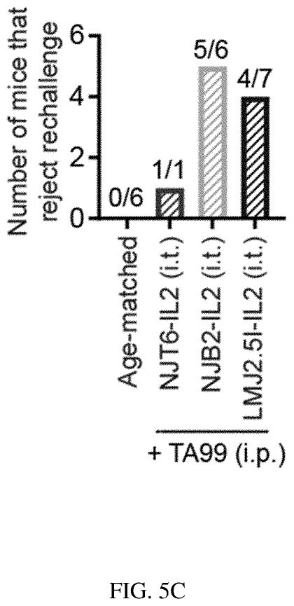

7-8 week old mice were inoculated with 1×10⁶ B16F10 cells subcutaneously (s.c.) in the right flank on day 0. Mice were treated on indicated days with 100 μg TA99 (i.p.) and 0.4 nmol (12.8 μg) of the following IL-2 fusions (i.t.): (1) NJB2-IL2 (based on parental nanobody NJB2), (2) LMJ2.5I-IL2, and (3) NJT6-IL2 (non-targeting control based on NJT6, a nanobody specific for the human TNC protein) (FIG. 5A). Tumor growth and survival were assessed (FIG. 5B).

Further, cured mice were rechallenged with 1×10⁵ B16F10 cells subcutaneously in the left flank on day 94 and tumor growth was monitored with no additional treatment. Rechallenge experiment was performed on N=1 for NJT6-IL2, N=6 for NJB2-IL2, and N=7 for LMJ2.5I-IL2. All tumors grew out in the naive age-matched controls (N=6) but increased survival is seen for the FN-EIIIB specific IL-2 immunocytokines (FIG. 5C).

### Example 5. Testing IL-12 Based Immunocytokines

IL-12 based immunocytokines were also tested. IL-12 immunocytokines specific for FN-EIIIB based on NJB2 lead to mild 4T1 growth delay after intravenous administration.

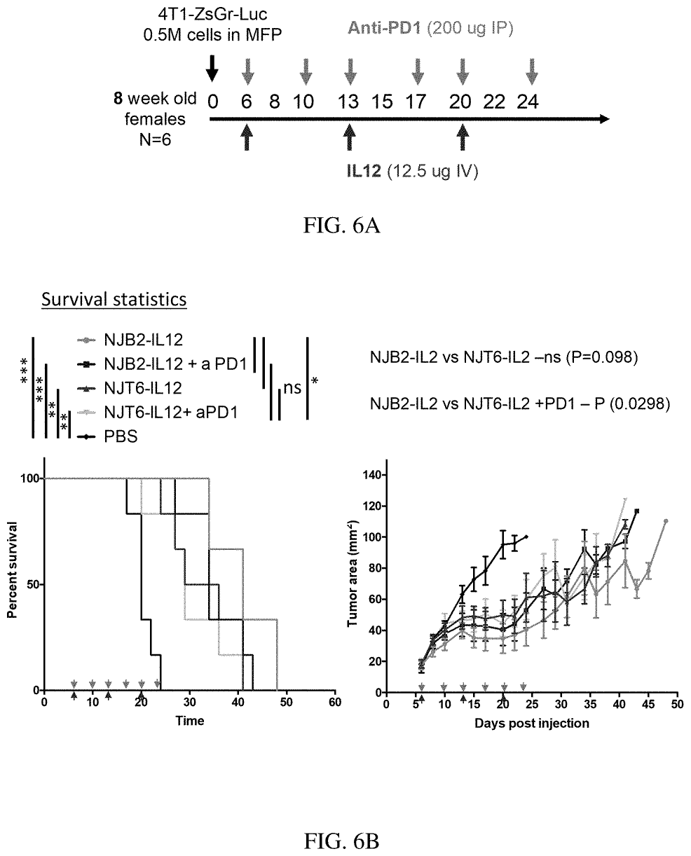

8-12 week old mice were inoculated with 0.5×10⁶ 4T1 cells in the mammary fat pad (MFP) on day 0 and treated on days indicated with 12.5 μg of the following nanobody IL-12 fusions (i.v.): (1) NJB2-IL12, (2) NJT6-IL2 (FIG. 6A). To see if antibodies to PD1 can potentiate the response to IL12, a group that is also treated with anti-PD1 was added for the IL12 fusions. Tumor growth and survival were assessed (FIG. 6B).

Mice treated with NJB2-IL12 lived longer than mice treated with NJT6-IL12. While the trends look promising, the extension was not statistically significant in this dosing scheme.

Sequences of IL12-nanobody fusions (IL12p40-linker-IL12p35-linker-nanobody-LPETGG-HHHHHH) are provided below.

IL12-LMJ1.2C:
(SEQ ID NO: 78)
MWELEKDVYVVEVDWTPDAPGETVNLTCDTPEEDDITWTSDQRHGVIGS
GKTLTITVKEFLDAGQYTCHKGGETLSHSHLLLHKKENGIWSTEILKNF
KNKTFLKCEAPNYSGRFTCSWLVQRNMDLKFNIKSSSSSPDSRAVTCGM
ASLSAEKVTLDQRDYEKYSVSCQEDVTCPTAEETLPIELALEARQQNKY
ENYSTSFFIRDIIKPDPPKNLQMKPLKNSQVEVSWEYPDSWSTPHSYFS
LKFFVRIQRKKEKMKETEEGCNQKGAFLVEKTSTEVQCKGGNVCVQAQD
RYYNSSCSKWACVPCRVRSGGSGGGSGGGSGGGSRVIPVSGPARCLSQS
RNLLKTTDDMVKTAREKLKHYSCTAEDIDHEDITRDQTSTLKTCLPLEL
HKNESCLATRETSSTTRGSCLPPQKTSLMMTLCLGSIYEDLKMYQTEFQ
AINAALQNHNHQQIILDKGMLVAIDELMQSLNHNGETLRQKPPVGEADP
YRVKMKLCILLHAFSTRVVTINRVMGYLSSAGGGGSGGGGSGGGGSQVQ
LVETGGGLVQAGGSLRLSCAASGSTFSHSAGGWYRQAPEKQRELVAGIR
SDGNINYADSVKDRFTISRDNASNTMYLQMNNLKPEDTAVYVCNIRGSY
GNTYYSRWGQGTQVTVSSGGLPETGGHHHHHH
IL12-LMJ2.5I:
(SEQ ID NO: 79)
MWELEKDVYVVEVDWTPDAPGETVNLTCDTPEEDDITWTSDQRHGVIGS
GKTLTITVKEFLDAGQYTCHKGGETLSHSHLLLHKKENGIWSTEILKNF
KNKTFLKCEAPNYSGRFTCSWLVQRNMDLKFNIKSSSSSPDSRAVTCGM
ASLSAEKVTLDQRDYEKYSVSCQEDVTCPTAEETLPIELALEARQQNKY
ENYSTSFFIRDIIKPDPPKNLQMKPLKNSQVEVSWEYPDSWSTPHSYFS
LKFFVRIQRKKEKMKETEEGCNQKGAFLVEKTSTEVQCKGGNVCVQAQD
RYYNSSCSKWACVPCRVRSGGSGGGSGGGSGGGSRVIPVSGPARCLSQS
RNLLKTTDDMVKTAREKLKHYSCTAEDIDHEDITRDQTSTLKTCLPLEL
HKNESCLATRETSSTTRGSCLPPQKTSLMMTLCLGSIYEDLKMYQTEFQ
AINAALQNHNHQQIILDKGMLVAIDELMQSLNHNGETLRQKPPVGEADP
YRVKMKLCILLHAFSTRVVTINRVMGYLSSAGGGGSGGGGSGGGGSRVQ
LVETGGGLVQAGGSLRLSCAVSGSTFSHSAGGWYRQAPEKQRELVAGIR
SDGNINYADSVKDRFTISRDNASNTMYLQMNNLKPEDTAVYVCNIRGSY
GNTYYSRWGQGAQVTVSSGGLPETGGHHHHHH
IL12-NJB2:
(SEQ ID NO: 80)
MWELEKDVYVVEVDWTPDAPGETVNLTCDTPEEDDITWTSDQRHGVIGS
GKTLTITVKEFLDAGQYTCHKGGETLSHSHLLLHKKENGIWSTEILKNF
KNKTFLKCEAPNYSGRFTCSWLVQRNMDLKFNIKSSSSSPDSRAVTCGM
ASLSAEKVTLDQRDYEKYSVSCQEDVTCPTAEETLPIELALEARQQNKY
ENYSTSFFIRDIIKPDPPKNLQMKPLKNSQVEVSWEYPDSWSTPHSYFS
LKFFVRIQRKKEKMKETEEGCNQKGAFLVEKTSTEVQCKGGNVCVQAQD
RYYNSSCSKWACVPCRVRSGGSGGGSGGGSGGGSRVIPVSGPARCLSQS
RNLLKTTDDMVKTAREKLKHYSCTAEDIDHEDITRDQTSTLKTCLPLEL
HKNESCLATRETSSTTRGSCLPPQKTSLMMTLCLGSIYEDLKMYQTEFQ
AINAALQNHNHQQIILDKGMLVAIDELMQSLNHNGETLRQKPPVGEADP
YRVKMKLCILLHAFSTRVVTINRVMGYLSSAGGGGSGGGGSGGGGSQVQ
LVETGGGLVQAGGSLRLSCAASGSTFSHNAGGWYRQAPEKQRELVAGIS
SDGNINYADSVKDRFTISRDNASNTMYLQMNNLKPEDTAVYVCNIRGSY
GNTYYSRWGQGTQVTVSSGGLPETGGHHHHHH
IL12-NJT6:
(SEQ ID NO: 81)
MWELEKDVYVVEVDWTPDAPGETVNLTCDTPEEDDITWTSDQRHGVIGS
GKTLTITVKEFLDAGQYTCHKGGETLSHSHLLLHKKENGIWSTEILKNF
KNKTFLKCEAPNYSGRFTCSWLVQRNMDLKFNIKSSSSSPDSRAVTCGM
ASLSAEKVTLDQRDYEKYSVSCQEDVTCPTAEETLPIELALEARQQNKY
ENYSTSFFIRDIIKPDPPKNLQMKPLKNSQVEVSWEYPDSWSTPHSYFS
LKFFVRIQRKKEKMKETEEGCNQKGAFLVEKTSTEVQCKGGNVCVQAQD
RYYNSSCSKWACVPCRVRSGGSGGGSGGGSGGGSRVIPVSGPARCLSQS
RNLLKTTDDMVKTAREKLKHYSCTAEDIDHEDITRDQTSTLKTCLPLEL
HKNESCLATRETSSTTRGSCLPPQKTSLMMTLCLGSIYEDLKMYQTEFQ
AINAALQNHNHQQIILDKGMLVAIDELMQSLNHNGETLRQKPPVGEADP
YRVKMKLCILLHAFSTRVVTINRVMGYLSSAGGGGSGGGGSGGGGSQVQ
LVETGGDLVQPGGSLRLSCAASGLTLDYYAIGWVRQAPGKEREGVSCIT
PQDGNTYYDDSVMGRFTILRDNAKNMVYLQMNNLKPEDTAVYFCAAAGA
LTLDPSEYEYWGQGTQVTVSSGGLPETGGHHHHHH

### Example 6. Testing IFNα Based Immunocytokines

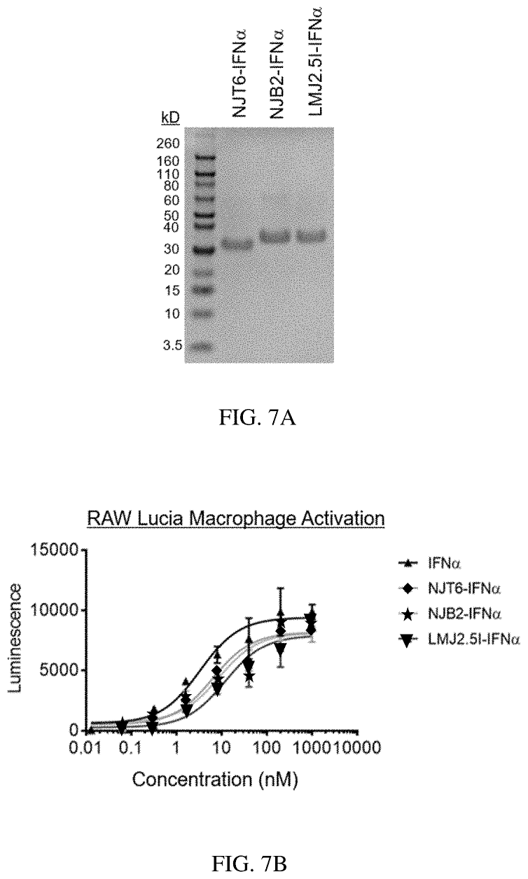

IFNα based immunocytokines were also tested. The following nanobody-IFNα fusions were purified: NJT6-IFNα, NJB2-IFNα, LMJ2.5I-IFNα (FIG. 7A). All IFNα fusions were active in vitro as tested by a RAW Lucia macrophage activation assay (FIG. 7B).

Sequences of nanobody-IFNα fusions (nanobody-linker-IFNα-HHHHHH) are provided below.

NJT6-IFNα:
(SEQ ID NO: 82)
QVQLVETGGDLVQPGGSLRLSCAASGLTLDYYAIGWVRQAPGKEREGVS
CITPQDGNTYYDDSVMGRFTILRDNAKNMVYLQMNNLKPEDTAVYFCAA
AGALTLDPSEYEYWGQGTQVTVSSGGGGGSCDLPQTHNLRNKRALTLLV
QMRRLSPLSCLKDRKDFRFPQEKVDAQQIQNAQAIPVLQELTQQVLNIF
TSKDSSAAWDASLLDSFCNDLHQQLNDLKACVMQEVGVQEPPLTQEDYL
LAVRTYFHRITVYLREKKHSPCAWEVVRAEVWRAMSSSAKLLARLSEEK
EHHHHHH
NJB2-IFNα:
(SEQ ID NO: 83)
QVQLVETGGGLVQAGGSLRLSCAASGSTFSHNAGGWYRQAPEKQRELVA
GISSDGNINYADSVKDRFTISRDNASNTMYLQMNNLKPEDTAVYVCNIR
GSYGNTYYSRWGQGTQVTVSSGGGGGSCDLPQTHNLRNKRALTLLVQMR
RLSPLSCLKDRKDFRFPQEKVDAQQIQNAQAIPVLQELTQQVLNIFTSK
DSSAAWDASLLDSFCNDLHQQLNDLKACVMQEVGVQEPPLTQEDYLLAV
RTYFHRITVYLREKKHSPCAWEVVRAEVWRAMSSSAKLLARLSEEKEHH
HHHH
LMJ2.5I-IFNα:
(SEQ ID NO: 84)
RVQLVETGGGLVQAGGSLRLSCAVSGSTFSHSAGGWYRQAPEKQRELVA
GIRSDGNINYADSVKDRFTISRDNASNTMYLQMNNLKPEDTAVYVCNIR
GSYGNTYYSRWGQGAQVTVSSGGGGGSCDLPQTHNLRNKRALTLLVQMR
RLSPLSCLKDRKDFRFPQEKVDAQQIQNAQAIPVLQELTQQVLNIFTSK
DSSAAWDASLLDSFCNDLHQQLNDLKACVMQEVGVQEPPLTQEDYLLAV
RTYFHRITVYLREKKHSPCAWEVVRAEVWRAMSSSAKLLARLSEEKEHH
HHHH

### REFERENCES

1. Jailkhani N, et al. (2019) Noninvasive imaging of tumor progression, metastasis, and fibrosis using a nanobody targeting the extracellular matrix. Proc Natl Acad Sci USA 116(28):14181-14190.
2. Chao G, et al. (2006) Isolating and engineering human antibodies using yeast surface display. Nat Protoc 1(2):755-768.
3. Tzeng A, Kwan B H, Opel C F, Navaratna T, & Wittrup K D (2015) Antigen specificity can be irrelevant to immunocytokine efficacy and biodistribution. Proc Natl Acad Sci USA 112(11):3320-3325.
4. Hansel T T, Kropshofer H, Singer T, Mitchell J A, & George A J (2010) The safety and side effects of monoclonal antibodies. Nat Rev Drug Discov 9(4):325-338.
5. De Vos J, Devoogdt N, Lahoutte T, & Muyldermans S (2013) Camelid single-domain antibody-fragment engineering for (pre)clinical in vivo molecular imaging applications: adjusting the bullet to its target. Expert Opin Biol Ther 13(8):1149-1160.
6. Hassanzadeh-Ghassabeh G, Devoogdt N, De Pauw P, Vincke C, & Muyldermans S (2013) Nanobodies and their potential applications. Nanomedicine (Lond) 8(6):1013-1026.
7. Rashidian M, et al. (2015) Noninvasive imaging of immune responses. Proc Natl Acad Sci USA 112(19):6146-6151.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean at least one than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which at least one limitation, element, clause, or descriptive term, from at least one of the claims or from at least one relevant portion of the description, is introduced into another claim. For example, a claim that is dependent on another claim can be modified to include at least one of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

In the claims, as well as in the specification above, all transitional phrases such as “comprising,” “including,” “carrying,” “having,” “containing,” “involving,” “holding,” “composed of,” are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood that, unless clearly indicated to the contrary, in any methods claimed herein that include more than one step or act, the order of the steps or acts of the method is not necessarily limited to the order in which the steps or acts of the method are recited.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present disclosure may be explicitly excluded from any at least one of the claims. Where ranges are given, any value within the range may explicitly be excluded from any at least one of the claims. Any embodiment, element, feature, application, or aspect of the compositions and/or methods of the disclosure, can be excluded from any at least one claims. For purposes of brevity, all of the embodiments in which at least one elements, features, purposes, or aspects is excluded are not set forth explicitly herein.

All references (e.g., published journal articles, books, etc.), patents and patent applications disclosed herein are incorporated by reference with respect to the subject matter for which each is cited, which, in some cases, may encompass the entirety of the document.

1. A nanobody that binds specifically to the EIIIB domain of fibronectin and comprises:
a) a complementarity-determining region (CDR) 1 comprising G(S/R/I/N)TFSH(N/S/Y/K)AG(G/S);
b) a CDR2 comprising (G/R)(I/V)(S/R/G/N)S(D/A/Y)GNI(N/S); and
c) a CDR3 comprising NIRG(S/T)YG(N/S)TYY(S/R)R,
wherein the nanobody does not comprise SEQ ID NO: 1.

2. The nanobody of claim 1, comprising:
a) a CDR1 selected from
(SEQ ID NO: 54)
GSTFSHNAGG,
(SEQ ID NO: 57)
GSTFSHSAGG,
(SEQ ID NO: 107)
GSTFSHYAGG,
(SEQ ID NO: 106)
GSTFSHKAGG,
(SEQ ID NO: 105)
GRTFSHNAGG,
(SEQ ID NO: 104)
GSTFSHNAGS,
(SEQ ID NO: 103)
GITFSHNAGG,
(SEQ ID NO: 63)
GRTFSHSAGG,
and
(SEQ ID NO: 102)
GNTFSHSAGG;
b) a CDR2 selected from
(SEQ ID NO: 55)
GISSDGNIN,
(SEQ ID NO: 58)
GIRSDGNIN,
(SEQ ID NO: 59)
GIGSDGNIN,
(SEQ ID NO: 60)
GISSAGNIN,
(SEQ ID NO: 108)
RISSDGNIN,
(SEQ ID NO: 109)
GINSYGNIN,
(SEQ ID NO: 62)
GIGSAGNIN,
(SEQ ID NO: 101)
GIRSDGNIS,
and
(SEQ ID NO: 61)
GVRSDGNIN;
and
c) a CDR3 selected from
(SEQ ID NO: 56)
NIRGSYGNTYYSR,
(SEQ ID NO: 100)
NIRGTYGNTYYSR,
(SEQ ID NO: 99)
NIRGSYGNTYYRR,
and
(SEQ ID NO: 98)
NIRGSYGSTYYSR.

3. The nanobody of claim 1, comprising a CDR1, CDR2, and CDR3 comprising:
a)
(SEQ ID NO: 57)
GSTFSHSAGG,
(SEQ ID NO: 58)
GIRSDGNIN,
and
(SEQ ID NO: 56)
NIRGSYGNTYYSR respectively;
b)
(SEQ ID NO: 57)
GSTFSHSAGG,
(SEQ ID NO: 59)
GIGSDGNIN,
and
(SEQ ID NO: 56)
NIRGSYGNTYYSR respectively;
c)
(SEQ ID NO: 57)
GSTFSHSAGG,
(SEQ ID NO: 60)
GISSAGNIN,
and
(SEQ ID NO: 56)
NIRGSYGNTYYSR respectively;
d)
(SEQ ID NO: 57)
GSTFSHSAGG,
(SEQ ID NO: 62)
GIGSAGNIN,
and
(SEQ ID NO: 56)
NIRGSYGNTYYSR respectively;
e)
(SEQ ID NO: 57)
GSTFSHSAGG,
(SEQ ID NO: 61)
GVRSDGNIN,
and
(SEQ ID NO: 56)
NIRGSYGNTYYSR respectively;
or
f)
(SEQ ID NO: 63)
GRTFSHSAGG,
(SEQ ID NO: 58)
GIRSDGNIN,
and
(SEQ ID NO: 56)
NIRGSYGNTYYSR respectively.

4. (canceled)

5. The nanobody of claim 1, further comprising a framework 1 comprising:
a)
(SEQ ID NO: 64)
QVQLVETGGGLVQAGGSLRLSCAAS;
b)
(SEQ ID NO: 68)
QVQLVGTGGGLVQAGGSLRLSCAAS;
c)
(SEQ ID NO: 85)
RVQLVETGGGLVQAGGSLRLSCAAS;
d)
(SEQ ID NO: 86)
QVRLVETGGGLVQAGGSLRLSCAVS;
e)
(SEQ ID NO: 66)
RVQLVETGGGLVQAGGSLRLSCAVS;
f)
(SEQ ID NO: 71)
QVQLVETGGGLVQAGGSLRLSCAVS;
g)
(SEQ ID NO: 72)
QVQLVETRGGLVQAGGSLRLSCAVS;
h)
(SEQ ID NO: 70)
QVQLVETEGGLVQAGGSLRLSCAAS;
i)
(SEQ ID NO: 87)
RVQLVETEGGLVQAGGSLRLSCAAS;
j)
(SEQ ID NO: 88)
QVRLVETGGGLVQAGGSLRLSCAAS;
k)
(SEQ ID NO: 73)
QVQLVEAGGGLVQAGGSLRLSCAAS;
l)
(SEQ ID NO: 89)
QVQLVETRGGLVQAGGSLRLSCAAS;
m)
(SEQ ID NO: 90)
RVQLVETRGGLVQAGGSLRLSCAAS;
or
n)
(SEQ ID NO: 91)
QVQLVGTGGGLVQAGGSLRLSCAVS.
and/or a framework 4 comprising:
a)
(SEQ ID NO: 65)
WGQGTQVTVSS;
b)
(SEQ ID NO: 69)
WGQGIQVTVSS;
c)
(SEQ ID NO: 92)
WSQGTQVTVSS;
d)
(SEQ ID NO: 97)
WGQRTQVTVSS;
e)
(SEQ ID NO: 93)
WGRGAQVTVSS;
f)
(SEQ ID NO: 67)
WGQGAQVTVSS;
g)
(SEQ ID NO: 94)
WGQGTQVTVFS;
h)
(SEQ ID NO: 95)
WGQGTRVTVSS;
or
i)
(SEQ ID NO: 96)
GGQGTQVTVFS.

6.-8. (canceled)

9. The nanobody of claim 1, wherein the nanobody comprises the amino acid sequence of any one of SEQ ID Nos. 2-53.

10. (canceled)

11. The nanobody of claim 1, wherein the nanobody is conjugated to a cargo molecule, optionally wherein the cargo molecule is linked to the N-terminus or C-terminus of the nanobody, further optionally, wherein the cargo molecule is a molecular imaging probe, a drug, a toxin, an antibody, a cytokine, an enzyme (e.g., ECM modifying enzyme), a CAR-T cell, a radioisotope, a protein or a nanoparticle.

12.-13. (canceled)

14. The nanobody of claim 11, wherein the cargo molecule is a cytokine selected from IL-2, IL-6, IL-8, IL-10, IL-12, IL-15, IL-18, TNF, IFNg, IFNb, IFNa, and a chemokine or mutants thereof.

15. The nanobody of claim 1, wherein the nanobody is attached to or associated with a carrier, optionally wherein the carrier further comprises a cargo molecule, and further optionally, wherein the carrier is a nanoparticle.

16.-18. (canceled)

19. A pharmaceutical composition comprising the nanobody of claim 1 and a pharmaceutically acceptable carrier.

20. A nucleic acid comprising a nucleic acid sequence encoding the nanobody of claim 1.

21. A vector comprising the nucleic acid of claim 20.

22. A cell comprising the nucleic acid of claim 20.

23. A method for producing a nanobody comprising (i) culturing the cell of claim 22 under conditions suitable for allowing the expression of the nanobody, and (ii) recovering the expressed nanobody.

24. A method of treating a disease or condition associated with fibronectin, comprising administering to a subject in need thereof an effective amount of the nanobody of claim 11, to deliver the cargo molecule to the extracellular matrix of the diseased tissue.

25. A method comprising administering an effective amount of the nanobody of claim 11 to a subject having a disease or condition associated with fibronectin, to deliver the cargo molecule to the extracellular matrix of the diseased tissue.

26.-28. (canceled)

29. The method of claim 24, wherein the disease or condition is a cancer, a cardiovascular disease, an inflammatory disorder, or fibrosis.

30. (canceled)

31. The method of claim 29, wherein the cancer is (i) melanoma, breast cancer or pancreatic cancer; or (ii) wherein the cancer is metastatic cancer.

32. (canceled)

33. A nanobody cytokine fusion protein, comprising a nanobody that binds specifically to the EIIIB domain of fibronectin fused to a cytokine, optionally wherein the cytokine is selected from IL-2, IL-6, IL-8, IL-10, IL-12, IL-18, TNF, IFN-γ, IFN-β, chemokines, and IFN-α and their mutants.

34. A nanobody cytokine fusion protein, comprising a nanobody of claim 1 fused to a cytokine, optionally wherein the cytokine is selected from IL-2, IL-6, IL-8, IL-10, IL-12, IL-18, TNF, IFN-γ, IFN-β, chemokines, and IFN-α and their mutants.

35. (canceled)

36. The nanobody cytokine fusion protein of claim 34 wherein the fusion protein comprises (i) at least two nanobodies fused to the cytokine optionally wherein the fusion protein comprises 2-25 nanobodies fused to the cytokine; or (ii) at least two cytokines fused to the nanobody, optionally wherein the at least two cytokines are attached to terminal ends of the nanobody.

37.-44. (canceled)
